(19) United States
(12) Patent Application Publication
(10) Pub. No.: US 2024/0382268 A1
(43) Pub. Date: Nov. 21, 2024

(54) SELF-STEERING ENDOLUMINAL DEVICE USING A DYNAMIC DEFORMABLE LUMINAL MAP

(71) Applicant: Magnisity Ltd., Hod-HaSharon (IL)

(72) Inventors: Ron BARAK, Tel-Aviv (IL); Benjamin GREENBURG, Hod-HaSharon (IL); Eyal KLEIN, Tel-Aviv (IL); Dror GARDOSH, Rishon LeTsiyon (IL)

(73) Assignee: Magnisity Ltd., Hod-HaSharon (IL)

(21) Appl. No.: 18/689,922

(22) PCT Filed: Sep. 8, 2022
(86) PCT No.: PCT/IL2022/050978

§ 371 Date: Mar. 7, 2024

Related U.S. Application Data
(60) Provisional application No. 63/340,512, filed on May 11, 2022.
(60) Provisional application No. 63/242,101, filed on Sep. 9, 2021.

(51) Int. Cl. A61B 34/20; A61B 1/005; A61B 1/267; A61B 5/16

(52) U.S. Cl. CPC A61B 34/20; A61B 1/0057; A61B 1/2676; A61B 5/165; A61B 2034/2065

(57) ABSTRACT

The present invention relates to a self-steering endoluminal system comprising an endoluminal device comprising a steerable elongated body and a computer memory storage medium, comprising one or more modules: a Navigational module comprising instructions for generating navigational actions to be performed by said steerable elongated body of said endoluminal device to reach a desired location as selected in a digital endoluminal map; a Deformation module comprising instructions for assessing potential deformations to one or more lumens caused by said navigational actions performed; a Stress module comprising instructions for assessing potential stress levels on said lumens caused by said navigational actions; and a High-level module comprising instructions to receive information from one or more of said Navigational module, Deformation module and Stress module and generate instructions to actuate, and optionally also actuate, said steerable elongated body of said endoluminal device accordingly.

### RELATED APPLICATION(S)

This application claims the benefit of priority of U.S. Provisional Patent Application No. 63/242,101 filed on Sep. 9, 2021, and from U.S. Provisional Patent Application No. 63/340,512 filed on May 11, 2022, the contents of which are incorporated herein by reference in their entirety.

### FIELD AND BACKGROUND OF THE INVENTION

The present invention, in some embodiments thereof, relates to a system and method for navigating one or more endoluminal devices and, more particularly, but not exclusively, to system and method for navigating one or more self-steering endoluminal devices.

In certain interventional procedures, in order to retrieve a biopsy sample or deliver localized treatment, a physician is required to reach specific targeted tissue inside the endoluminal structure, for example the lung bronchial tree, or for example the cerebral vascular system, or for example in the digestive system. To accomplish this, it is standard practice to use an endoluminal tool, for example a bronchoscope in the lung, or for example a catheterization kit for vascular systems, which is manually guided through the bifurcated lumen according to real-time direct visual imaging, such as direct vision, or such as angiogram, which is a cumbersome task, especially in cases where the target is in a peripheral location and/or the path to reach the location is a tortuous path. One of the apparent difficulties of driving a tool in such cases is a mechanical one. For example, in the lungs, standard bronchoscopes are usually relatively thick (for example, 6 mm in diameter) compared to the peripheral airways into which they need to be forced (for example, 1 mm in diameter). Another problem relates to navigation: in a direct visualization navigation the physician must understand the bronchoscope's location in the lungs based on video imaging only. However, due to the airways' fractal nature, as the airways become smaller and smaller it is often difficult to distinguish one airway from the other making it probable for even experienced physicians to erroneously choose a wrong turn, eventually not reaching the desired target.

Likewise, in vascular systems, for example cerebral vascular systems, or for example hepatic vascular systems, the structure is delicate, narrowing, and tortuous, and usage of standard angiograms to guide microcatheters and guidewires is challenging and require years of training and specialization.

In recent years it has become more common for bronchoscopists to use Navigational Bronchoscopy for peripheral interventions in the lung. Such procedures are performed using systems which usually provide 2D and/or 3D navigational renders of the lung, based on a CT or other near-real-time imaging, on to which a reference of the location of the instrument is displayed.

Thus, such a system assists the physician in guiding an instrument such as bronchoscope, endoscope or a general catheter (with or without a camera) to the targeted location. Such guided instruments have a benefit of usually being smaller in diameter relative to a standard bronchoscope (for example, 3-4 mm, or less). Such an instrument usually has a working channel (for example, of diameter 2 mm or greater) wide enough to allow the physician to introduce biopsy and/or treatment tools to the targeted tissue, once reaching the desired location inside the anatomy.

Additional background art includes European patent EP2849669B1 disclosing a medical system comprising a processor and a surgical device including a tracking system disposed along a length of an elongate flexible body. The processor receives a first model of anatomic passageways of a patient anatomy. The first model includes a set of model passageways representing proximal and distal branches. The processor also receives from the tracking system a shape of the elongate flexible body positioned within the proximal and distal branches. The processor determines, based on the shape of the elongate flexible body, a set of forces acting on the patient anatomy in response to the surgical device positioned within the proximal and distal branches. The processor also generates a second model by deforming the first model based on the set of forces and display the second model and a representation of the elongate flexible body within the second model.

U.S. patent Ser. No. 10/499,993B2 disclosing a processing system comprising a processor and a memory having computer readable instructions stored thereon. The computer readable instructions, when executed by the processor, cause the system to receive a reference three-dimensional volumetric representation of a branched anatomical formation in a reference state and obtain a reference tree of nodes and linkages based on the reference three-dimensional volumetric representation. The computer readable instructions also cause the system to obtain a reference three-dimensional geometric model based on the reference tree and detect deformation of the branched anatomical formation due to anatomical motion based on measurements from a shape sensor. The computer readable instructions also cause the system to obtain a deformed tree of nodes and linkages based on the detected deformation, create a three-dimensional deformation field that represent the detected deformation of branched anatomical, and apply the three-dimensional deformation field to the reference three dimensional geometric model.

U.S. patent Ser. No. 10/610,306B2 disclosing a method that comprises determining a shape of a device positioned at least partially within an anatomical passageway. The method further comprises determining a set of deformation forces for a plurality of sections of the device, where determining the set of deformation forces comprises determining a stiffness of each section of the plurality of sections of the device. The method further comprises generating a composite model indicating a position of the device relative to the anatomical passageway based on: the shape of the device, the set of deformation forces, including an effect of each section of the plurality of sections on a respective portion of the anatomical passageway, and anatomical data describing the anatomical passageway.

U.S. patent Ser. No. 10/524,641B2 disclosing a navigation guidance which is provided to an operator of an endoscope by determining a current position and shape of the endoscope relative to a reference frame, generating an endoscope computer model according to the determined position and shape, and displaying the endoscope computer model along with a patient computer model referenced to the reference frame so as to be viewable by the operator while steering the endoscope within the patient.

U.S. Patent Application No. 20180193100A1 disclosing an apparatus comprising a surgical instrument mountable to a robotic manipulator. The surgical instrument comprises an elongate arm. The elongate arm comprises an actively controlled bendable region including at least one joint region, a passively bendable region including a distal end coupled to the actively controlled bendable region, an actuation mechanism extending through the passively bendable region and coupled to the at least one joint region to control the actively controlled bendable region, and a channel extending through the elongate arm. The surgical instrument also comprises an optical fiber positioned in the channel. The optical fiber includes an optical fiber bend sensor in at least one of the passively bendable region or the actively controlled bendable region.

U.S. Pat. No. 9,839,481B2 disclosing a system that comprises a handpiece body configured to couple to a proximal end of a medical instrument and a manual actuator mounted in the handpiece body. The system further includes a plurality of drive inputs mounted in the handpiece body. The drive inputs are configured for removable engagement with a motorized drive mechanism. A first drive component is operably coupled to the manual actuator and also operably coupled to one of the plurality of drive inputs. The first drive component controls movement of a distal end of the medical instrument in a first direction. A second drive component is operably coupled to the manual actuator and also operably coupled to another one of the plurality of drive inputs. The second drive component controls movement of the distal end of the medical instrument in a second direction.

U.S. Pat. No. 9,763,741B2 disclosing an endoluminal robotic system that provides the surgeon with the ability to drive a robotically-driven endoscopic device to a desired anatomical position in a patient without the need for awkward motions and positions, while also enjoying improved image quality from a digital camera mounted on the endoscopic device.

U.S. Patent Application No. US20110085720A1 disclosing registration between a digital image of a branched structure and a real-time indicator representing a location of a sensor inside the branched structure is achieved by using the sensor to “paint” a digital picture of the inside of the structure.

Once enough location data has been collected, registration is achieved. The registration is “automatic” in the sense that navigation through the branched structure necessarily results in the collection of additional location data and, as a result, registration is continually refined.

### SUMMARY OF THE INVENTION

Following is a non-exclusive list including some examples of embodiments of the invention. The invention also includes embodiments which include fewer than all the features in an example and embodiments using features from multiple examples, also if not expressly listed below.

Example 1. A method of generating a steering plan for a self-steering endoluminal system, comprising:
a. selecting a location accessible through one or more lumens in a digital endoluminal map to which a self-steering endoluminal device needs to reach;
b. generating navigational actions for said endoluminal device to reach said location;
c. assessing potential deformations to one or more lumens caused by said navigational actions performed by said endoluminal device;
d. updating said steering plan according to a result of said assessing potential deformations while said self-steering endoluminal system is reaching said location.
   Example 2. The method according to example 1, further comprising performing said navigational actions until reaching said location.
   Example 3. The method according to example 1 or example 2, wherein said updating said steering plan is performed in real-time.
   Example 4. The method according to any one of examples 1-3, wherein said method further comprise assessing potential stress levels on said lumens caused by said navigational actions performed by said endoluminal device.
   Example 5. The method according to example 4, wherein said method is performed until said potential stress levels are below a predetermined threshold.
   Example 6. The method according to any one of examples 1-5, further comprising providing said plan to said self-steering endoluminal system.
   Example 7. The method according to any one of examples 1-6, further comprising generating said digital endoluminal map comprising said one or more lumens based on an image.
   Example 8. The method according to example 7, wherein said image is a CT scan.
   Example 9. The method according to example 7, wherein said image is an angiogram.
   Example 10. The method according to any one of examples 1-9, wherein said generating navigational actions comprises running a first simulation of said navigational actions.
   Example 11. The method according to any one of examples 1-10, wherein said assessing potential deformations comprises running a second simulation of said potential deformations.
   Example 12. The method according to example 11, further comprising updating said digital endoluminal map according to said potential deformations simulated in said second simulation.
   Example 13. The method according to example 4, wherein said assessing potential stress levels comprises running a simulation of said potential stress levels.
   Example 14. The method according to example 13, further comprising updating said navigational actions to cause a reduction in said potential stress levels.
   Example 15. The method according to any one of examples 1-14, wherein said assessing potential deformations further comprises assessing deformation caused by breathing, heartbeats and other external causes.
   Example 16. A self-steering endoluminal system, comprising:
a. an endoluminal device comprising a self-steerable elongated body;
b. a computer memory storage medium, comprising one or more modules, comprising:
   i. a Navigational module comprising instructions for generating navigational actions to be performed by said steerable elongated body of said endoluminal device to reach a desired location as selected in a digital endoluminal map;
   ii. a Deformation module comprising instructions for assessing potential deformations to one or more lumens caused by said navigational actions performed by said steerable elongated body of said endoluminal device;
   iii. a High-level module comprising instructions to receive information from one or more of said Navigational module and said Deformation module and actuate said steerable elongated body of said endoluminal device accordingly.
   Example 17. The system according to example 16, wherein said computer memory storage medium further comprises a Stress module comprising instructions for assessing potential stress levels on said lumens caused by said navigational actions performed by said steerable elongated body of said endoluminal device.
   Example 18. The system according to example 17, wherein said High-level module further comprises instructions to receive information from said Stress module and actuate said steerable elongated body of said endoluminal device accordingly.
   Example 19. The system according to any one of examples 16-18, wherein said endoluminal device comprises one or more sensors for monitoring a location of said endoluminal device during said navigational actions.
   Example 20. The system according to example 19, further comprising an external transmitter for allowing said monitoring.
   Example 21. The system according to any one of examples 16-20, wherein said Navigational module comprises instructions for generating navigational actions to be performed by said steerable elongated body of said endoluminal device to aid reaching a desired location as selected in a digital endoluminal map.
   Example 22. The system according to any one of examples 16-21, wherein said High-level module further comprises instructions to generate a steering plan based on said received information.
   Example 23. The system according to any one of examples 16-22, wherein said High-level module further comprises instructions to generate said digital endoluminal map comprising said one or more of lumens based on an image.
   Example 24. The system according to example 23, wherein said image is a CT scan.
   Example 25. The system according to example 23, wherein said image is an angiogram.
   Example 26. The system according to any one of examples 16-25, wherein said Navigational module further comprises instructions for running a first simulation of said navigational actions.
   Example 27. The system according to any one of examples 16-26, wherein said Deformation module further comprises instructions for running a second simulation of said potential deformations.
   Example 28. The system according to example 27, further comprising updating said digital endoluminal map according to said potential deformations simulated in said second simulation.
   Example 29. The system according to example 17, wherein said Stress module further comprises instructions for running a third simulation of said potential stress levels.
   Example 30. The system according to example 29, further comprising updating said navigational actions to cause a reduction in said potential stress levels.
   Example 31. The system according to any one of examples 16-30, wherein said assessing potential deformations further comprises assessing deformation caused by breathing, heartbeats and other external causes.
   Example 32. The system according to any one of examples 16-31, wherein said endoluminal device comprises one or more steering mechanisms comprising one or more pull wires, one or more pre-curved shafts, one or more shafts having variable stiffness along a body of said one or more shaft and one or more coaxial tubes.
   Example 33. The system according to example 32, wherein one or more of said one or more pre-curved shafts and one or more shafts having variable stiffness along a body of said one or more shaft are one within another.
   Example 34. The system according to example 32, wherein said one or more steering mechanisms are configured to cause one or more steering actions comprising rotation of the shaft, advancing/retracting the shaft, deflection of the tip of the device and deflection of a part of the shaft of the device.
   Example 35. A method of generating a steering plan for a self-steering endoluminal system, comprising:
a. selecting a location accessible through one or more lumens in a digital endoluminal map to which a self-steering endoluminal device needs to reach;
b. generating navigational actions for said endoluminal device to reach said location;
c. assessing potential deformations to one or more lumens caused by said navigational actions performed by said endoluminal device;
d. assessing potential stress levels on said lumens caused by said navigational actions performed by said endoluminal device;
e. performing steps b-d until said potential stress levels are below a predetermined threshold.
   Example 36. The method according to example 35, further comprising providing said plan to said self-steering endoluminal system.
   Example 37. The method according to example 35, further comprising generating said digital endoluminal map comprising said one or more lumens based on an image.
   Example 38. The method according to example 37, wherein said image is a CT scan.
   Example 39. The method according to example 37, wherein said image is an angiogram.
   Example 40. The method according to example 35, wherein said generating navigational actions comprises running a first simulation of said navigational actions.
   Example 41. The method according to example 35, wherein said assessing potential deformations comprises running a second simulation of said potential deformations.
   Example 42. The method according to example 41, further comprising updating said digital endoluminal map according to said potential deformations simulated in said second simulation.
   Example 43. The method according to example 35, wherein said assessing potential stress levels comprises running a simulation of said potential stress levels.
   Example 44. The method according to example 43, further comprising updating said navigational actions to cause a reduction in said potential stress levels.
   Example 45. The method according to example 35, wherein said assessing potential deformations further comprises assessing deformation caused by breathing, heartbeats and other external causes.
   Example 46. A self-steering endoluminal system, comprising:
a. an endoluminal device comprising a steerable elongated body;
b. a computer memory storage medium, comprising one or more modules, comprising:
   i. a Navigational module comprising instructions for generating navigational actions to be performed by said steerable elongated body of said endoluminal device to reach a desired location as selected in a digital endoluminal map;
   ii. a Deformation module comprising instructions for assessing potential deformations to one or more lumens caused by said navigational actions performed by said steerable elongated body of said endoluminal device;
   iii. a Stress module comprising instructions for assessing potential stress levels on said lumens caused by said navigational actions performed by said steerable elongated body of said endoluminal device;
   iv. a High-level module comprising instructions to receive information from one or more of said Navigational module, Deformation module and Stress module and actuate said steerable elongated body of said endoluminal device accordingly.
   Example 47. The system according to example 46, wherein said endoluminal device comprises one or more sensors for monitoring a location of said endoluminal device during said navigational actions.
   Example 48. The system according to example 47, further comprising an external transmitter for allowing said monitoring.
   Example 49. The system according to example 46, wherein said Navigational module comprises instructions for generating navigational actions to be performed by said steerable elongated body of said endoluminal device to aid reaching a desired location as selected in a digital endoluminal map.
   Example 50. The system according to example 46, wherein said High-level module further comprises instructions to generate a steering plan based on said received information.
   Example 51. The system according to example 46, wherein said High-level module further comprises instructions to generate said digital endoluminal map comprising said one or more of lumens based on an image.
   Example 52. The system according to example 51, wherein said image is a CT scan.
   Example 53. The system according to example 51, wherein said image is an angiogram.
   Example 54. The system according to example 46, wherein said Navigational module further comprises instructions for running a first simulation of said navigational actions.
   Example 55. The system according to example 46, wherein said Deformation module further comprises instructions for running a second simulation of said potential deformations.
   Example 56. The system according to example 55, further comprising updating said digital endoluminal map according to said potential deformations simulated in said second simulation.
   Example 57. The system according to example 46, wherein said Stress module further comprises instructions for running a third simulation of said potential stress levels.
   Example 58. The system according to example 57, further comprising updating said navigational actions to cause a reduction in said potential stress levels.
   Example 59. The system according to example 46, wherein said assessing potential deformations further comprises assessing deformation caused by breathing, heartbeats and other external causes.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced. In the drawings:

FIG. 1 is a schematic representation of an exemplary endoluminal system, according to some embodiments of the invention;

FIG. 2 is a schematic representation of an exemplary endoluminal device, according to some embodiments of the invention;

FIG. 3a is a schematic representation of an exemplary digital/virtual 3D volumetric image provided to the NavNN, according to some embodiments of the invention;

FIG. 3b is a schematic representation of an exemplary digital/virtual 3D volumetric image including camera sensor images provided to the NavNN, according to some embodiments of the invention;

FIGS. 4a-e are schematic representations of exemplary sequence of driving actions based on real-time localization images, as generated in real-time during procedure and processed by the NavNN module, according to some embodiments of the invention;

FIG. 5 is a schematic representation of an exemplary volumetric tessellation of a catheter using 3D pyramid primitives, according to some embodiments of the invention;

FIGS. 6a-b are schematic representation of exemplary 3D localization images centered according to different objects, according to some embodiments of the invention;

FIGS. 7a-b are schematic representations of exemplary non-deformed and deformed localization images, according to some embodiments of the invention;

FIG. 8 is a flowchart of an exemplary method of displaying correct 2D/3D system views to reflect the lumen deformation, according to some embodiments of the invention;

FIGS. 9a-d are schematic representations of exemplary actions performed by the DeformNN module, according to some embodiments of the invention;

FIG. 10 is a schematic representation of an exemplary endoluminal device with the tracking and navigational system, according to some embodiments of the invention; and

FIG. 11 is a flowchart of an exemplary method of use of the system, according to some embodiments of the invention.

### DESCRIPTION OF SPECIFIC EMBODIMENTS OF THE INVENTION

The present invention, in some embodiments thereof, relates to a system and method for navigating one or more endoluminal devices such as for example an endoscope, or for example a miniaturized endoluminal robotic device, or for example an endovascular catheter, or for example an endovascular guidewire; and, more particularly, but not exclusively, to system and method for navigating one or more self-steering endoluminal devices. In some embodiments, when more than one devices are used, they are navigated contemporarily. While in the following paragraphs a single device will be used to explain the invention, it should be understood that the same explanations apply also to multiple devices being used at the same time.

In some embodiments, to display a position of the endoluminal device on a navigational map, the instrument is tracked in real-time or near real-time. In some embodiments, various methods can be used for the localization of the instrument and displaying its position on the navigational map, including electromagnetic single-sensor, multi-sensor, fiber optics, fluoroscopic visualization, and others. For example, in some embodiments, the instrument has a single tracking sensor (for example, electromagnetic sensor) at the catheter's tip, providing a 6-DOF position and orientation (also referred to “location”, which hereinafter means both position and orientation) to the navigation system. The terms “catheter”, “endoscope” and “endoluminal device” mean the same, which is a device used inside lumens, and are used herein interchangeably. The term “navigational map” mean a representation of the anatomy, which may be based on various modalities or detection methods, including CT, CTA, angiograms, MR scans, Ultrasonography, 3D ultrasound reconstructions, fluoroscopic imaging, tomosynthesis reconstructions, OCT, and others. In some embodiments, the tip's location is registered with the patient's anatomy and displayed in navigational 2D/3D views. The term “registration” refers to the process of transforming different sets of data into one coordinate system, unless otherwise specified. In some embodiments, the physician can therefore see a representation of the catheter's tip as it lies, for example, inside the lungs, or for example, inside cerebral vascularity, and manipulate the catheter to the desired target, which is usually also displayed in the presented views. In some embodiments, the catheter's shape is sensed using a “shape sensor”, which may be based on fiber optics. In some embodiments, the catheter's shape is monitored using other means, for example using RFID technology, which do not require active transmission from within the endoluminal device to allow the monitoring of the device, or by reconstructing its 3D shape using one or more fluoroscopic projections in near real-time. In some embodiments, reconstructing a device's 3D shape from fluoroscopic projections is performed by identifying the device's tip and/or full curve in multiple fluoroscopic 2D projections, identifying the fluoroscope's location in some reference coordinate system, for example using optical fiducials, and finding the device's 3D location and/or shape by means of optimization, such that the back-projected 2D device's curves will fit the observed 2D curves from the fluoroscopic projections. In some embodiments, the catheter's shape is registered to the patient's anatomy and presented to the physician in 2D/3D views. In some embodiments, the catheter may include multiple position sensors (for example, electromagnetic) to enable tracking of the full catheter's position and absolute shape relative to some referenced transmitter. In some embodiments, the catheter may not include any sensors. In some embodiments, it may be a passive catheter which is visible under fluoroscopy. In some embodiments, the catheter's shape is being tracked using fluoroscopy by reconstruction methods using one or more fluoroscopic projections. In some embodiments, the catheter's shape and location is then registered to the patient's anatomy and being displayed to the physician. In some embodiments a combination of these methods is used.

In some embodiments, various 2D/3D views are used to display the location of the catheter in relation to the navigational map. In some embodiments, the views are used by the physician to decide how to manipulate the catheter such that it will reach the target. In some embodiments, optionally, a pre-planned path from the entry point to the target is displayed in these views. In some embodiments, during the intervention, following the path, physician articulates the catheter tip and drives it closer to the target, while watching the real-time tracked movement of the instrument on the displayed view.

In some embodiments, various mechanisms can be used to drive the instrument to the desired location. In some embodiments, the mechanisms are driven manually, operated by the physician, with one or more levers providing articulation of the catheter's tip. In some embodiments, the catheter may be manually inserted with a fixed curve at the distal end. In some embodiments, the catheter is mounted to a robotic driving mechanism, controlled by a remote-control panel. In some embodiments, at any time, and particularly when reaching the desired target, the robotic driving mechanism may fix the catheter in space or in anatomy, eliminating the need to hold the catheter and allowing stable insertion of tools via the working channel without changing the catheter's position and orientation. In some embodiments, a potential advantage of fixing the catheter in anatomy is that, since in some cases it is not enough to fix the catheter “in space”, since the anatomy moves relative to any fixed point in space (for example, when the patient breathes), it is potentially beneficial to fix the catheter “in anatomy”, that is, move it automatically in space to retain its position relative to an anatomical target regardless of patient's motion/breathing or tissue movement, deflection or deformation.

There are many cases where pushing an instrument beyond the luminal wall may be harmful, or at least requires caution. With a manual instrument, as it is pushed against a barrier, a resistive force is being propagated back to the catheter's handle, and from there is sensed by the physician. A trained physician is aware of the risk and therefore operates the catheter cautiously: when the resistive force grows beyond what the physician judges to be excessive, the physician may relive the pressure and pull the catheter. Once the catheter is retracted the physician may alter the tip orientation and slowly push it forward towards the target. However, with a mechanically, electro-mechanically and/or power driven catheter, or in a case of a very long catheter, the physician is unable to sense these forces, increasing the risk of harming the patient or damaging the catheter. Therefore, in some embodiments, the system comprises a mechanism to replace the lost natural force feedback, for example, by force sensors and mechanical tracking.

### Overview

An aspect of some embodiments of the invention relates to a system and method for navigating an endoluminal device, for example a bronchial endoscope, or for example an endovascular device such as guidewire, or such as micro-catheter, or such as catheter or such as emboli retrieval tool, or such as coiling tool, using a virtual dynamic deformable luminal map. In some embodiments, the navigation is performed automatically by the system using a self-steering endoluminal device. In some embodiments, the navigation, and the updating of the navigation is performed in real time while the endoluminal device is advancing towards a desired location. In some embodiments, the deformation is tracked in real-time by a deformation-aware tracking system, as the product of the real-time tracking of the full location and/or shape of the endoluminal device inside the patient and translated into the virtual dynamic deformable map. In some embodiments, an informative 3D Localization Image is generated in real-time from the fully tracked endoluminal device or from a plurality of fully tracked endoluminal devices and the virtual dynamic deformable map, including the current real-time position and full shape of the device. In some embodiments, the localization image encodes all information needed for a qualified human and/or an intelligent machine (AI) to decide on the best driving action, for example steering, forward motion or backward motion, required in any location in order to reach the target. In some embodiments, the localization image can be processed by a Navigational Neural Network (NavNN) module to produce an intelligent driving action. In some embodiments, a non-deformed localization image may be initially used to find the deformation using a Deformation Neural Network (DeformNN) module, therefore generating a deformed localization image for navigation. In some embodiments, the system and/or the method are versatile and can be used, for example, to perform a complete autonomous navigation from beginning to end, or in another example, the navigation may be broken into smaller human supervised steps, for example controlled by an intuitive “Tap-to-drive” user interface, in which autonomous navigation is performed, for example, from the current position to an indicated position (for example, “tapped” on a touch screen interface) in the anatomy. In some embodiments, the system and/or the method may be used to display recommended navigational instructions for a human physician. In some embodiments, the system and/or the method may be used in a self-steering endoscope, whereas the endoscope's tip automatically aligns with the path to target and the physician only advances the tip distally or proximally, along the patient's airways. In some embodiments, the system and/or the method may be used with any endovascular device, such as catheter, guidewire, tool, or other, fitted with a driver apparatus with self-steering capabilities, whereas the driver apparatus causes the endovascular device tip to automatically align with a pre-planned path, so that the physician is only required to advance the tip distally or proximally, inside the blood vessel, wither manually or using the driver apparatus. In some embodiments, the system and/or the method are suitable for collecting training data to enhance AI performance (for example to teach one or more neural network modules, as will be further explained below). In some embodiments, the autonomous driving actions are supervised by additional safety mechanisms, ensuring safe manipulation of the device in the body.

An aspect of some embodiments of the invention relates to a system that rasterizes 3D pyramid primitives onto a 3D render target and uses it for rendering real-time 3D localization image in a navigational procedure. In some embodiments, optionally, the method is implemented in a GPU ASIC/FPGA. In some embodiments, optionally, the method is exposed to a developer through an OpenGL extension or with DirectX. In some embodiments, optionally, the method is used for rendering real-time 3D composite data for processing by 3D Neural Network. In some embodiments, optionally, the method is used for rendering real-time 3D image of a tracked hand and fingers.

An aspect of some embodiments of the invention relates to a system and/or a method for encoding and optionally displaying navigational data in a 3D multi-channel localization image. Optionally in a virtual 3D multi-channel localization image. In some embodiments, optionally, one of the channels contains a segmented lumen structure. In some embodiments, optionally, the segmented lumen structure is binary. In some embodiments, optionally, the segmented lumen structure is a scalar likelihood map. In some embodiments, optionally, the segmented lumen structure is deformed using a Deformation Neural Network module. In some embodiments, optionally, the segmented lumen structure is represented by its skeleton. In some embodiments, optionally, one of the channels contains raw CT data, raw MRI data, raw angiogram data and any combination thereof. In some embodiments, optionally, one or more channels contain a catheter in its estimated position inside the body. In some embodiments, optionally, the catheter is represented as a full or partial curve. In some embodiments, optionally, the catheter is represented only by its tip. In some embodiments, optionally, the catheter is rendered in its deformed position inside the anatomy. In some embodiments, optionally, the catheter is rendered in its non-deformed position inside the anatomy. In some embodiments, optionally, one of the channels contains the pathway to target. In some embodiments, optionally, one of the channels contains the segmented target. In some embodiments, optionally, one of the channels contains the target sphere. In some embodiments, optionally, one of the channels contains images of an endoscopic camera. In some embodiments, optionally, the images are 2D and rendered in the 3D localization image using back-projection along corresponding rays. In some embodiments, optionally, the images contain a depth channel and are rendered in the 3D localization image as a 3D surface using their depth channel. In some embodiments, optionally, the localization image has a special position and alignment. In some embodiments, optionally, the localization image is centered at the catheter's tip. In some embodiments, optionally, the localization image is centered at the pathway. In some embodiments, optionally, the localization image is centered at the closest pathway point. In some embodiments, optionally, the localization image is aligned with the catheter's tip direction. In some embodiments, optionally, the localization image's X axis is aligned with the catheter's tip direction. In some embodiments, optionally, the localization image's X axis is aligned with the pathway direction. In some embodiments, optionally, the localization image's Z axis is aligned with the normal vector of the next bifurcation. In some embodiments, optionally, the 3D localization image input is generated in real-time. In some embodiments, optionally, the localization image is rendered using 3D pyramid tessellation techniques. In some embodiments, optionally, the segmented lumen structure is rendered in real-time in its deformed state, as computed by a deformation-aware localization system. In some embodiments, optionally, the segmented lumen structure is rendered in real-time in its deformed state using a Deformation Neural Network. In some embodiments, optionally, the catheter position is rendered in its position, as computed by a tracking system. In some embodiments, optionally, the catheter's position is rendered in its anatomical deformation-compensated position using a Deformation Neural Network module.

An aspect of some embodiments of the invention relates to a system and/or a method for producing automatic navigational driving actions. In some embodiments, optionally, a localization image is processed using Navigational Neural Network (NavNN) module. In some embodiments, optionally, the localization image is processed using a 3D Convolutional Neural Network (3D CNN). In some embodiments, optionally, the localization image is processed using a 3D Recurring Neural Network (3D RNN). In some embodiments, optionally, the localization image includes a camera channel to produce better driving actions. In some embodiments, optionally, the NavNN possesses memory. In some embodiments, optionally, the NavNN carries a state vector between predictions. In some embodiments, optionally, a high-level module operates the NavNN. In some embodiments, optionally, the high-level module chooses the best driving action by selecting the maximal output of the NavNN. In some embodiments, optionally, the high-level module automatically activates motors based on the NavNN output. In some embodiments, optionally, the high-level module periodically generates random driving actions to add exploration to the navigation and evade local extremum points of the NavNN output. In some embodiments, optionally, the high-level module automatically rolls (rotates) the catheter on certain predetermined time intervals. In some embodiments, optionally, hysteresis is used on the NavNN output to prevent “jumping” between different output driving actions. In some embodiments, optionally, safety mechanisms are enforced on the NavNN output to prevent harmful driving actions. In some embodiments, optionally, the catheter is not pushed if a certain force is exerted on the patient. In some embodiments, optionally, the catheter is automatically pulled back if a certain force is exerted on the patient. In some embodiments, optionally, the exerted force is computed by analyzing the full catheter curve inside the segmented lumen structure. In some embodiments, optionally, the exerted force is sensed by force sensors in the catheter's handle or along the catheter's body. In some embodiments, optionally, the NavNN is trained in a supervised training using 3D localization image inputs, labeled with their corresponding driving actions. In some embodiments, optionally, the labeled samples are generated using a realistic simulator module. In some embodiments, optionally, the labeled samples are collected from real robotic navigational procedures. In some embodiments, optionally, the labeled samples are collected from real manual navigational procedures. In some embodiments, optionally, the operator's manual driving actions are classified automatically. In some embodiments, optionally, the driving actions are classified using a proximal and distal catheter sensor. In some embodiments, optionally, the catheter's handle contains one or more sensors for classifying the operator's actions. In some embodiments, optionally, the NavNN is trained in an unsupervised training using 3D localization image inputs. In some embodiments, optionally, the NavNN is trained in a realistic simulator module using reinforcement learning.

An aspect of some embodiments of the invention relates to a system and/or a method for finding the anatomical position of a catheter inside a deformed luminal structure (for more explanations on “deformed luminal structure”—see below). In some embodiments, optionally, the localization image is processed using Deformation Neural Network (DeformNN) module. In some embodiments, optionally, the localization image is processed using a 3D CNN. In some embodiments, optionally, the localization image is processed using a 3D RNN. In some embodiments, optionally, the localization image is processed using a 3D U-Net. In some embodiments, optionally, the localization image includes a camera channel to improve accuracy. In some embodiments, optionally, the DeformNN possesses memory. In some embodiments, optionally, the DeformNN carries a state vector between predictions. In some embodiments, optionally, the DeformNN outputs the image of a deformed-compensated lumen structure. In some embodiments, optionally, the DeformNN outputs the image of a catheter in its anatomical position inside the input lumen structure. In some embodiments, optionally, the DeformNN outputs the image of one or more hypothetical catheters in their anatomical positions inside the lumen structure, with their corresponding confidence levels. In some embodiments, optionally, the DeformNN outputs a single probability per catheter reflecting the confidence in the input catheter in its position in the input lumen structure. In some embodiments, optionally, a deformation of the luminal structure is searched such that it maximizes the output probability of the DeformNN. In some embodiments, optionally, a high-level module operates the DeformNN. In some embodiments, optionally, in the case of outputting a deformed lumen structure, the input and output lumen structures are registered to compute deformation vectors. In some embodiments, optionally, in the case of outputting a deformed catheter curve, the input and output catheters are registered to compute deformation vectors. In some embodiments, optionally, the deformation vectors are applied to the full lumen structure or to the catheter position to display deformation-compensated system views. In some embodiments, optionally, the partial localization image output of the DeformNN is rigged with the missing channels and inputted to the NavNN to produce automatic driving actions. In some embodiments, optionally, the DeformNN is trained in a supervised training using 3D localization image inputs, labeled with their corresponding deformation-compensated output images. In some embodiments, optionally, the labeled samples are generated using a realistic simulator module. In some embodiments, optionally, deformation of the lumen structure is simulated in the simulator module using realistic deformation models. In some embodiments, optionally, deformation of the lumen structure is simulated in the simulator using polynomial, spline or rigid 3D transformations. In some embodiments, optionally, the labeled samples are collected from real manual navigational procedures. In some embodiments, optionally, one or more catheters are inserted into known anatomical positions (for example, in peripheral locations) and the anatomy is deformed by applying internal and external forces, to record deformation of the lumen structure. In some embodiments, optionally, trackable sensors are placed inside the organ to record the deformation. In some embodiments, optionally, multiple CBCT (Cone-beam CT) scans are performed and registered using deformable registration to compute deformation vectors. In some embodiments, optionally, the DeformNN is further trained on the luminal structure of a specific patient prior to procedure.

An aspect of some embodiments of the invention relates to a system and/or a method for displaying multiple catheter hypotheses in a navigational procedure. In some embodiments, optionally, two or more catheter hypotheses are displayed inside the lumen structure on a 2D/3D view with different opacity or intensity based on their confidence levels. In some embodiments, optionally, a single catheter is displayed until the position where it splits in different direction of different hypotheses. In some embodiments, optionally, the shared segment of the catheter hypotheses is displayed normally, while the split segments are displayed in different color, intensity or opacity. In some embodiments, optionally, upon catheter position ambiguity, the screen splits into multiple independent displays of different catheter hypotheses. In some embodiments, optionally, falling back to a single winning hypothesis, the winning half-screen “pushes” the losing half-screen out of view.

An aspect of some embodiments of the invention relates to a system and/or a method for computing a force risk estimate of a catheter inside a luminal structure. In some embodiments, optionally, the force risk estimate is computed using the catheter's fully tracked position inside the lumen structure. In some embodiments, optionally, the force risk estimate is computed by estimating contact forces and inner catheter forces. In some embodiments, optionally, the force risk estimate is computed using StressNN by providing a 3D localization image which visualizes the catheter inside the lumen structure. In some embodiments, optionally, the StressNN is trained with labeled samples which are generated using a realistic simulator module. In some embodiments, optionally, the force risk estimate is computed in the simulator module using physically simulated force estimates.

An aspect of some embodiments of the invention relates to a system and method of self-steering optionally wireless and optionally disposable endoluminal devices using real-time 3D localization images. In some embodiments, optionally, the device is wirelessly paired with a patient in a pre-procedure pairing process. In some embodiments, optionally, the patient's data (segmented lumen structure, target planning, etc.) is transferred to the device using NFC or any other wireless method. In some embodiments, optionally, the device applies deformation compensation to the segmented lumen structure or to the catheter. In some embodiments, optionally, the deformation compensation is done using a skeletal deformation model and optimization methods. In some embodiments, optionally, the deformation compensation is done using DeformNN. In some embodiments, optionally, the device uses NavNN to produce accurate automatic driving actions and feedbacks. In some embodiments, optionally, the device automatically rotates the catheter (especially useful when utilizing passive J-catheters) using miniature motors in the handle to align the catheter with the pathway to target, based on NavNN outputs. In some embodiments, optionally, the device automatically pushes or pulls the endoluminal portion of the device using miniature actuators, for example in the handle, to advance the device in either way in relation to the target. In some embodiments, optionally, the device uses LED or a vibration motor feedbacks to instruct the operator during navigation. In some embodiments, optionally, the device is handheld, and the push/pull actions are carried by the operator, per the device's instructions. In some embodiments, optionally, the device is mounted into a robotic driving mechanism and is driven autonomously without human's mechanical intervention. In some embodiments, optionally, the automatic navigation is stopped based on a force risk estimate.

An aspect of some embodiments of the invention relates to a system and/or a method for controlling driven endoluminal devices by indicating a destination. In some embodiments, the driving function is achieved for example by using an electromechanical apparatus. In some embodiments, the endoluminal device is advanced in the lumen using other driving methods, for example by applying magnetic fields to a magnet-fitted device, or for example by using pneumatic or hydraulic pressure to actuate a device, or other methods. In some embodiments, optionally, an operator causes the tip of an instrument to be navigated to a position in the anatomy by indicating the desired end-position and orientation of the instrument tip. In some embodiments, optionally, the destination is marked by tapping on a point in a 3D map representing the organ, displayed on a touchscreen. In some embodiments, optionally, the destination is marked by clicking a mouse pointer on a location on a computer screen displaying an anatomical imaging, for example a CT slice, or for example an angiogram, or for example a sonogram or for example a MRI. In some embodiments, optionally, the destination is marked by choosing from a menu or other user interface (UI) element a predetermined position. In some embodiments, optionally, the destination is automatically suggested by the system. In some embodiments, optionally, the destination is indicated by issuing a voice command. In some embodiments, optionally, the destination is indicated on a multi-waypoint curved planar view map, which resembles a progress bar. In some embodiments, optionally, waypoints are obtained by performing limited maneuvers in sequential order according to their order on the map. In some embodiments, optionally, a “magnifying glass” view is used for indicating an exact destination in the targeted area. In some embodiments, optionally, a “first person” view is used for indicating an exact destination in the targeted area. In some embodiments, optionally, the system is triggered to stop the advance according to predetermined maximum travelled distance. In some embodiments, optionally, a dead-man-switch is used to stop the motion of the device. In some embodiments, optionally, a “stabilize in anatomy” mechanism is used to actively prevent the tip from crossing a determined proximity to a determined structure, using motorized micro movements and adjustments.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Endoluminal System

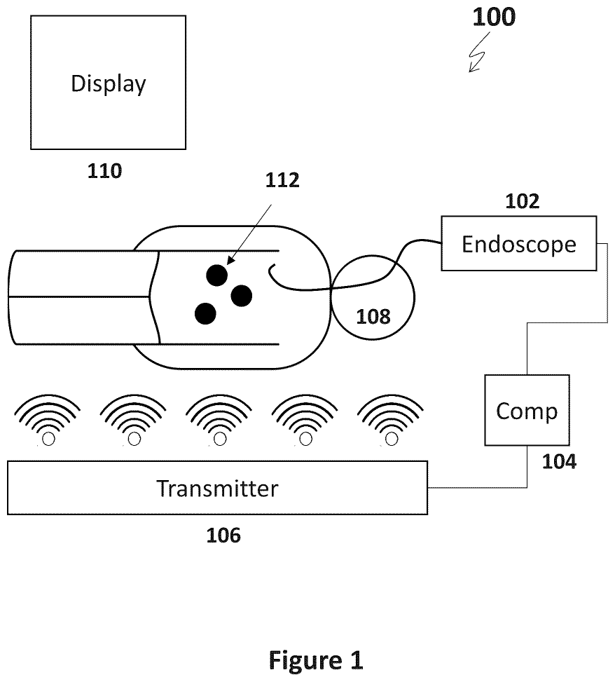

Referring now to FIG. 1, showing a schematic representation of an exemplary endoluminal system, according to some embodiments of the invention. In some embodiments, the endoluminal system 100 comprises an endoluminal device 102, for example an endoscope, configured for endoluminal interventions. In some embodiments, the endoluminal device 102 is connected to a computer 104 configured to monitor and control actions performed by the endoluminal system 100, including, in some embodiments, self-steering actions of the endoluminal device 102. In some embodiments, the endoluminal system 100 further comprises a transmitter 106 configured to generate electromagnetic fields used by the endoluminal system 100 to monitor the location of the endoluminal device 102 inside the patient 108. In some embodiments, the endoluminal system 100 further comprises a display unit 110 configured to show dedicated images to the operator, which potentially assist the operator during the navigation of the endoluminal device 102 during the endoluminal interventions. In some embodiments, the endoluminal system 100 optionally further comprises one or more sensors 112 configured to monitor movements of the patient 108 during the endoluminal intervention. In some embodiments, the patient's movements are used to assist in the navigation of the endoluminal device 102 inside the patient 108. Each of the abovementioned exemplary parts of the endoluminal system 100, and exemplary methods thereof, will be further explained below.

### Exemplary Endoluminal Devices 102 and Tracking Systems Thereof

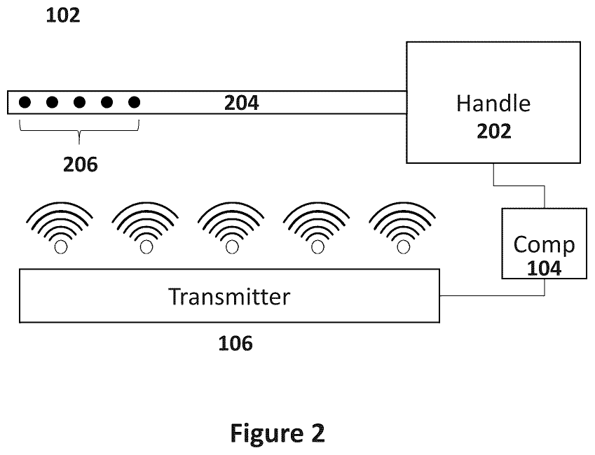

Referring now to FIG. 2, showing a schematic representation of an exemplary endoluminal device, according to some embodiments of the invention. In some embodiments, the endoluminal system 100 comprises an endoluminal device 102, for example an endoscope. In some embodiments, the endoluminal device 102 comprises a handle 202 and an elongated body 204. In some embodiments, the endoluminal device 102 comprises a plurality of sensors 206 along the elongated body 204 configured to detect transmission signals from the transmitter 106. In some embodiments, the endoluminal system 100 monitors the location of the elongated body 204 using the plurality of sensors 206.

In some embodiments, the plurality of sensors 206 are one or more of a 3-axis accelerometer, a 3-axis gyroscope, a 3-axis magnetometer. In some embodiments, the plurality of sensors 206 are digital sensors. In some embodiments, the plurality of sensors 206 are analog sensors comprising an additional A2D element in order to transmit the sensed analog data in a digital data form. In some embodiments, the plurality of sensors 206 are a combination of digital sensors and analog sensors.

In some embodiments, the elongated body 204 comprises a flexible printed circuit board (PCB) within and/or placed along elongated body 204. Additional information can be found in International application publication No. WO2021048837, its contents are incorporated herein in entirely. In some embodiments, the PCB is communicationally connected to a microcontroller, for example by a same data bus that includes few wire lines, for example two to four wires. For example, an inter-integrated circuit (I2C) is used as a digital connection interface between microcontroller and the plurality of sensors 206 installed along the flexible PCB. In some embodiments, the endoluminal device only requires two wires for exchange of data between the sensors and microcontroller. In some embodiments, a potential advantage of having such a small number of wires is that is allows to keep a small wire count in catheters that are needed to be kept small.

In some embodiments, the flexible PCB may have eight, five, ten, or any suitable number of sensors installed thereon, for example all connected to the same I2C bus (for example serial data and serial clock lines). In some embodiments, the microcontroller is connected to the flexible PCB using a 4-wires shielded cable, for example including voltage and/or ground wires. In some embodiments, the microcontroller provides the voltage and/or ground for digital sensors, for example in addition to the two data lines for the readings of digital measurements by sensors. In some embodiments, the microcontroller reads the sensors, for example, sequentially and/or simultaneously and sends the sensor readings to the computer 104, for example over wired and/or wireless communication.

In some embodiments, the design of the flexible PCB and/or positioning of the sensors thereon provides the positions and/or orientations of the sensors, for example, when PCB is straight. In some embodiments, for example, during the manufacturing process, the PCB is attached inside and/or along the elongated body 204, for example in a manner that determines the positions and/or orientations of the sensors 206, for example, with respect to the elongated body 204. In some embodiments, the computer 104 may be calibrated with the initial 6DOF orientation and/or position of the sensors, for example 6DOF orientation and/or position of the sensors when the elongated body 204 is straight. In some embodiments, the initial 6DOF orientation and/or position data, along with information about rigidity and/or flexibility limitations of the elongated body 204, is incorporated in the catheter localization algorithm as shape constraints. In some embodiments, for example, based on incorporated shape constraints, two neighboring sensors cannot point to opposite directions. In some embodiments, a potential advantage of utilizing shape constrains in the calculations is that it potentially provides a more sophisticated localization algorithm, which takes shape constraints into account, and potentially enables the system 100 to be both compact and robust. In some embodiments, solving for the 6DOF position and/or orientation of all the sensors while imposing physical shape constraints on the elongated body's 204 full-curve shape potentially reduces the number of parameters of the motion model and thus, for example, potentially preventing over-fitting of the measured data. In some embodiments, an additional potential advantage of using the shape constraints, is the computer 104 may refrain from erroneously calculating a position and/or orientation of any sensor due to a noisy or distorted measurement, because the position and/or orientation solution must comply, for example, with position and/or orientation solutions of neighboring sensors, for example so they would together describe a smooth, physically plausible elongated body 204.

In some embodiments, the computer 104 takes into account dynamic electromagnetic distortion by incorporating the distortion in the localization algorithm, for example in order to provide accurate solutions. Different methods used to compensate for dynamic magnetic distortions were explained in International Patent Publication WO2021048837, which its content is incorporated herein by reference entirely.

### Exemplary Method of Monitoring the Endoluminal Device

As mentioned above, exemplary methods of monitoring the endoluminal device were described elsewhere, for example, in International Patent Publication N. WO2021048837, the contents are incorporated herein by reference entirely.

In short, in some embodiments, the computer 104 receives from the transmitter 106 data about a momentary phase of a generated alternating electromagnetic field. In some embodiments, the computer 104 receives a sensed value of local magnetic field, sensed by the plurality of sensors 206 along the elongated body 204 that senses the magnetic field generated by transmitter 106. In some embodiments, the plurality of sensors 206 sense the generated magnetic field in its local coordinate system, therefore, in some embodiments, the magnetic field reading is rotated according to its orientation with respect to transmitter 106. In some embodiments, the computer 104 then associates between the transmitter data and the sensed magnetic field from the sensors. In some embodiments, the computer 104 then calculates the position and orientation of the plurality of sensors that provided a sensed magnetic field value, for example the 6DOF or 5DOF localization of each of sensors and/or an overall position, orientation and/or curve of the elongated body 204, based on the transmitter data and the sensed magnetic field from the sensors. In some embodiments, optionally, the computer 104 uses for the localization calculations accelerometer and/or gyroscope readings of corresponding sensors included in the plurality of sensors 206. In some embodiments, the electromagnetic field frequency of transmitter 106 is constrained to from about 10 Hz to about 100 Hz. Optionally to from about 10 Hz to about 200 Hz. Optionally to from about 10 Hz to about 500 Hz. Optionally to from about 10 Hz to about 1000 Hz. In some embodiments, the computer 104 utilizes a mathematical model to describe the motion of the elongated body 204. In some embodiments, the computer 104 tracks each of the plurality of sensors 206 independently. For example, the computer 104 is configured to predict the state of each of the plurality of sensors 206 of a next timeframe, for example based on the state in a current timeframe, and/or based on an Inertial Measurement Unit (TMU) (that provides information of the device's motion and pose) bundle measurements that may be used to correct the prediction. In some embodiments, the computer 104 utilizes, in its catheter localization algorithm, known structural relationships between the plurality of sensors 206 to calculate an estimation of the position, orientation and/or curve of the elongated body 204 as a whole, for example rather than calculating position and/or orientation for each of the plurality of sensors 206 separately.

### Exemplary Principal of the Advanced Monitoring System

In some embodiments, the invention relates to a system that utilizes an advanced monitoring system to provide guiding and, in some embodiments, automatic steering (further explained below), to an endoluminal device.

During a navigational bronchoscopy procedure, the physician needs to choose the best driving action to perform on a catheter depends on the data presented in the system views in order to advance the catheter closer towards the target. Both with a handheld as well as robotic driving mechanism, the physician tries to manipulate the catheter's handle (e.g., push/pull/roll/bend) either manually or remotely to “improve” the state of the catheter as presented in the views. The term “state” in this context, refers to the relative location of the device according to a predetermined path towards a desired location inside the body of the patient. The more “on track” the device is, the better is the “state” of the device in relation to the desired target location. As an easy example, when the catheter is located before the main carina (the first carina connecting the left and right lungs), the physician needs to articulate the catheter's tip in the correct direction (e.g., by roll/bend) and push the catheter down to the left lung, supposing the desired target is located in the left lung. After doing so the catheter would be displayed in the left lung in the real-time system views, so that the catheter's state is in fact improved. Alternatively, if the physician mistakenly pushes the catheter down the right lung then the catheter will be displayed in the right lung, further away from the pathway to the desired target as displayed in the system views, so that the catheter state was worsen. The physician, noticing the catheter is now further from the pathway to target, would pull the catheter back and renavigate to the correct lung, so as to improve the catheter's state in relation to the destination target.

Being able to estimate the catheter's state in relation to the destination target and the target's pathway may seem like a trivial task but may prove very tricky whenever the state of the catheter, map or orientation are not perfectly known. Multiple reasons exist for these crucial factors to be unknown, most notably dynamic deformations of the tissue. Dynamic deformations of the tissue may be caused by many forces, organic or inorganic. For example, bending the catheter may exert forces on the tissue and cause a dynamic deformation, moving the airways along with the catheter. It should be noted that some systems are unable to compensate for this dynamic deformation. In these systems the displayed airways map is fixed from the beginning of the procedure, not accounting for changes due to breathing, dynamically applied forces during procedure (such as in the described case), anesthesia induced atelectasis, heart movement, pneumothorax, etc.

Even when the decision is made according to perfect map conditions, problems with executing the maneuver may hinder navigation, requiring trial-and-error repetition. For example, frictional forces may prevent the catheter from advancing down the desired airway. It is then up to a skilled physician to carefully interact with the catheter, watching for real-time catheter behavior in the system views, to try and alter the orientation of the catheter's tip in different directions, pull and push the catheter until the catheter is advanced in the correct airway towards the target. In another example the physician may wish to advance the catheter towards the upper lobes. To do so, the catheter's tip must be articulated in a sharp angle. However, when the catheter is pushed it may slide and proceed forward towards the middle lobe, missing the turn. A skilled physician would then pull back and try different levels of bending, with different orientations (some of them are nontrivial) and speeds for advancing the catheter until managing to enter the upper lobe.

As seen by the examples above, the problem of choosing the best driving actions is nontrivial, and not always in agreement with straightforward geometrical reasoning. What may seem like the optimal choice of action from a geometrical point-of-view can prove to be non-beneficial in practice, and different actions need to be iterated by a skilled physician until the catheter is correctly advanced. It is also important to note that in all the examples provided above it was assumed that the physician is displayed by the system with perfect 2D/3D views which provide the physician with full real-time understanding of the 3D state of the catheter relative to the surrounding 3D airways and pathway to target. However, views are inherently limited by the fact that the human eye can only sense a 2D projected image, for example, as presented by 2D monitors. Occasionally, stereoscopic views are being generated and displayed to each eye separately using special headsets or glasses which create the effect of 3D perception, however, the data being displayed is still 2D in nature —it is only a 2D projection (or multiple projections) of the raw 3D data, as observed by a virtual camera in the 3D world. Since the system views are essentially 2D projections of the raw 3D data, they may suffer from problems such as occlusion (for example, one airway occluding another from the virtual camera's point-of-view), misperception of depth (for example, the distance between two features seems much smaller in the projected view than it really is in practice) among others. To overcome this, views are designed so that the skilled physician would be able to “complete the picture” using imagination and 3D perception: for example, to overcome occlusions the virtual camera may be placed in an optimal position with minimal occlusions using an automatic camera positioning algorithm, also by automatically moving the camera the viewer gets the perception of 3D positions to a certain extent. However, the final understanding of the true 3D structure of the displayed features (e.g., the catheter, the surrounding airways and the pathway to target) depends on the 3D perception capabilities of a skilled physician, which makes the system less usable for common users.

In some embodiments, the system of the invention comprises a self-steering endoscope, which for example can be handheld. In some embodiments, the physician holds the endoscope and slides it down the patient's airways. In some embodiments, the endoscope's tip steers automatically to align with the next bifurcation, such that the physician would only need to push the endoscope forward, optionally at a certain and predetermined velocity. In some embodiments, the endoscope's automatic steering is powered by a Navigational Neural Network (NavNN) module, which is fed with the virtual dynamic localization image and produces output driving actions/commands.

See below for further explanations regarding the NavNN.

In some embodiments, the roll and deflection driving commands are translated into mechanical manipulations using miniature motors or other actuators inside the endoscope's handle. In some embodiments, optionally, the user is then given navigational feedbacks (for example, push/pull back) and, with the aid of the NavNN, the user is allowed to reach the desired target safely and easily. In some embodiments, optionally, the catheter may be mounted to a fully robotic driving mechanism and be navigated to a target with a tap-to-drive user interface. In some embodiments, instead of manually operating the catheter with a remote controller, the physician is provided a screen which displays the catheter in its position along a pathway to target. In some embodiments, the physician then taps the next closest bifurcation or waypoint along the pathway and the robot, based on the outputs from the NavNN, performs the required driving actions in order to advance the catheter from its current position to the next waypoint. In some embodiments, the performed maneuver is relatively short and can be supervised by the physician operator. In some embodiments, once reaching the next waypoint, the physician then instructs the robot to perform the next maneuvers sequentially until reaching the target. In some embodiments, the physician may instruct the robot to perform two consecutive maneuvers automatically, or do all remaining maneuvers to reach the target, in a complete autonomous navigation scenario.

In some embodiments, the system further comprises a Catheter Stress Detection algorithm, which uses the fully tracked catheter's position and shape in its anatomical position to estimate catheter stress inside the patient's lumen, represented using a force risk estimate.

See below for further explanations regarding the Catheter Stress Detection algorithm.

In some embodiments, the algorithm examines the catheter's shape and provides alerts such as in cases where the catheter is about to break or starts to apply excessive forces on the airways. In some embodiments, these alerts can be used to supervise the robotic driving maneuvers as well as provide alerts in the handheld case for patient safety and system stability. In some embodiments, the algorithm can be based on pure geometrical considerations as well as a dedicated Stress Neural network (StressNN).

See below for further explanations regarding the Stress Neural Network (StressNN).

In some embodiments, while force sensors may be integrated inside the driving mechanism to predict the forces applied by the device to the lumen (as done by a physician with a handheld catheter), another option is to utilize device tracking information relative to advance distance performed by the robot to estimate the device's stress inside the lumen. In some embodiments, as described herein, a device's fully tracked curve is analyzed, in its localized state inside the anatomy, to accurately predict the level of stress of the device inside the lumen. Generally, when the device follows a smooth path it is most likely relieved and cannot harm the tissue. As the device starts to build a curvy shape inside rather straight lumen, and as loops are starting to form, the device's stress level is considered high and the robotic driving mechanism is stopped. In some embodiments, in those cases, the device is then pulled and relieved, or in other cases an alert is triggered. In some embodiments, a potential advantage of combining the proposed stress detection mechanism with external or internal force sensors is that it potentially provides a fuller protection for a robotically driven catheter.

In some embodiments, optionally, in order to increase the accuracy of the system, the virtual luminal map used for navigation is actively deformed according to the real-time deformation of the luminal structure. In some embodiments, a potential advantage of deforming the virtual luminal map is that it potentially avoids displaying the device in its wrong anatomical position, potentially even outside of the luminal boundaries, which can result in erroneous navigational decisions. In some embodiments, the deformation is tracked in real-time by a deformation-aware tracking system, which is for example based on a skeletal model of the luminal structure. In some embodiments, the skeletal model is deformed using optimization methods under certain shape constraints as to find the true position of the fully tracked device in the deformed anatomy. In some embodiments, the deformation of the luminal structure is found in real-time based on the device's fully tracked position using a dedicated Deformation Neural Network (DeformNN) module based on many training samples.

See below for further explanations regarding the Deformation Neural Network (DeformNN) module.

In some embodiments, the NavNN module is given the most accurate deformation-compensated localization image, whether generated by a dedicated Deformation Neural Network based on a non-deformed localization image or by the product of a general deformation-aware tracking system, for deciding on the best driving action.

### Exemplary Generation of the Virtual/Digital Dynamic Deformable Luminal Map

In some embodiments, as mentioned above, the system navigates the device inside the body of the patient utilizing a virtual/digital dynamic deformable luminal map. In some embodiments, as a beginning step in the generation of the virtual/digital dynamic deformable luminal map, the system is provided, for example, with a CT image (or a MRI image or an angiogram, etc.) of the patient in question, or for example an angiogram. In some embodiments, the system is configured to analyze the image and generate a virtual/digital 3D volumetric image of the patient. In some embodiments, the virtual/digital 3D volumetric image is the image used by the system to perform the navigation. In some embodiments, the digital 3D volumetric image is the image provided to the Navigational Neural Network (NavNN) module and/or the Deformation Neural Network (DeformNN) module and/or the Stress Neural Network (StressNN) module.

In some embodiments, during the procedure the system is configured to correlate between the actual measured locations of the catheter inside the patient and incorporate those measured locations into the virtual/digital 3D volumetric image.

### Exemplary Navigational Neural Network (NavNN) Module

In some embodiments, a Navigational Neural Network (NavNN) module is provided and “sees” a real-time system view (3D Localization Image) and decides on the best driving action based on this view. In some embodiments, however, instead of being displayed with the 2D projected views, as the user is, the localization image encodes all relevant navigational information as raw 3D data. In some embodiments, the system is configured to overcome the inherent problems of displaying 2D or 3D images to a human user to allow that user to analyze and decide which path to take by allowing the NavNN module to analyze the relevant information as raw 3D data (the human user cannot process 3D raw data). In some embodiments, this information does not suffer from 2D projection problems such as occlusion and depth misperception, which happens to human users. In some embodiments, the NavNN processes the data in 3D based on trained weights and produces output driving actions. For example, each NN contains “weights” such as convolutional filter coefficients, threshold, etc. In some embodiments, these weights are found during the training process of the NN and are used for further predictions through the model. In some embodiments, these actions are then displayed to the user as driving recommendations (for example, but not limited: (a) PUSH shaft (catheter) forward/PULL back, (b) ROTATE shaft (catheter) clockwise/counterclockwise, (c) DEFLECT joint #1 or deflecting segment #1 up/down/right/left, (d) ROTATE joint #2 clockwise/counterclockwise, (e) DEFLECT joint #3 or deflecting segment #3 up/down/right/left etc.), or be automatically used in an autonomous or semi-autonomous navigation system. In some embodiments, the NavNN is trained on data from a physical realistic simulation module (see below) or on annotated recordings using supervised or unsupervised methods. For example, a physical simulation mimics realistic endoluminal navigational procedures. For example, the simulation may show all 2D/3D views available to a user during navigational bronchoscopy, except that the displayed tracked endoscope is not real, instead it is a physically simulated virtual endoscope placed inside a patient's CT scan (or MRI scan, or angiogram, etc.). In some embodiments, all interactions between the endoscope and the patient are simulated physically in software.

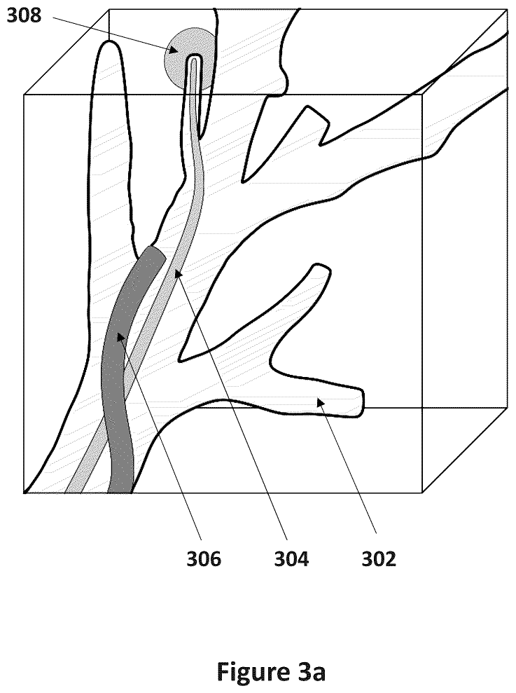

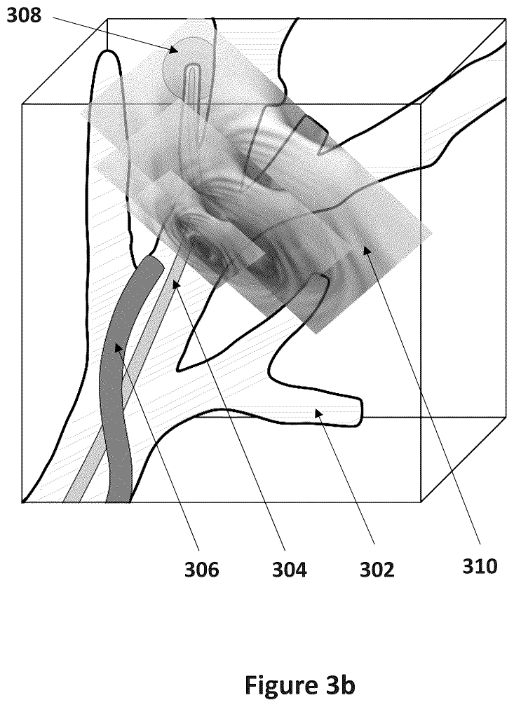

Referring now to FIG. 3a, showing a schematic representation of an exemplary digital/virtual 3D volumetric image provided to the NavNN, according to some embodiments of the invention. In some embodiments, as explained above, the localization image provided to the NavNN is a digital/virtual 3D volumetric image of a certain resolution and scale derived, for example, from a preoperative CT of the patient (or MRI scan, or angiogram, etc.). In some embodiments, for example, the image may be a 100×100×100 multi-channel voxels image, where each voxel is a cube sized 0.5 mm³, such that the image covers a total spatial volume of 5×5×5 cm³.

In some embodiments, each of the channels in the localization image represents a different navigational feature. In some embodiments, for example, the first channel represents the segmented luminal structure 302 (as mentioned, derived from the preoperative CT/MRI/angiogram/etc. of the patient), the second channel represents the pathway to the target 304 and the third channel represents the full catheter curve 306 (inside the localization image box of the region of interest (ROI), in this case only a single catheter is being used) as being tracked by the real-time tracking system, as depicted in FIG. 3a. Optionally, not shown in FIG. 3a, a fourth channel is added with the preoperative raw (unsegmented) CT data (or MRI data, or angiogram data, etc.). In some embodiments, a potential advantage of providing the raw unsegmented CT data (or MRI data, or angiogram data, etc.) is that it potentially enables the NavNN to base its navigational decisions not only on segmented airway structure, but also on non-segmented airways which may be present in the CT scan and traversed by the catheter. In some embodiments, instead of using a binary segmentation image, the first channel 302 representing the luminal structure may contain a scalar image which reflects the likelihood of each voxel being inside a lumen, for example as outputted by a lumen segmentation Neural Network or by any other non-binary lumen segmentation algorithm. In some embodiments, in this case, the NavNN module is presented with richer information describing the full lumen structure, including very small lumen tubes which would have been potentially dropped by applying a binary threshold on the segmentation. In some embodiments, the NavNN module can then base its navigational decisions not only on binary segmented airway structure, but on “soft-segmented” airways (ones with small likelihood) as well. In some embodiments, optionally, the second channel 304 also includes the segmented target or a spherical target 308 at the end of the pathway to target, or the target is included in a dedicated separate channel. In some embodiments, optionally, the first channel 302 represents the skeleton of the segmented luminal structure, where the value of each skeleton voxel may be equal the radius of the segmented luminal structure at the voxel.

Referring now to FIG. 3b, showing a schematic representation of an exemplary digital/virtual 3D volumetric image including camera sensor images provided to the NavNN, according to some embodiments of the invention. In some embodiments, optionally, a fifth channel 310 may be added containing data from an imaging sensor located at the catheter's tip, as shown for example in FIG. 3b. In some embodiments, the image may be a 2D frame, for example, of VGA resolution (640×480 pixels). In some embodiments, since the frame is 2D, but the localization image is 3D, it is important to specify how to render the 2D frame inside the 3D localization image in sensible positions that will result in effective use of the camera images by the NN in its training and prediction, as explained below. In some embodiments, since the depth of each pixel (meaning the distance of each pixel from the camera sensor) is usually unknown, it may be located on any point along a ray which extends from the 3D camera position (which is known due to 3D tracking of the catheter) in a 3D direction determined by that pixel (according to its x, y position inside the camera sensor). In some embodiments, each 2D pixel is rendered using back-projection along a complete ray, starting at the 3D camera position and extending in the 3D direction of that pixel from the camera forward to space, until colliding with the localization image's boundaries, as illustrated for example in FIG. 3b. In some embodiments, when a depth channel is available for the camera images (for example, by using stereoscopic cameras or by 3D reconstruction techniques or by LiDAR or by any other suitable method), the depth values are used to render each camera pixel in its exact 3D position in space, resulting in a 2D surface rendered in 3D, instead of back-projecting each pixel along a complete ray. In some embodiments, a potential advantage of combining imaging sensor data inside the 3D localization image is that it can potentially improve the NavNN performance. In some embodiments, the NavNN module is configured to identify luminal passageways in the image (relative to the catheter's 3D position in space) and improve its output driving actions by using the identified lumens. It should be noted that, in some embodiments, the order of channels is unimportant for the NavNN, as long as it is consistent between training and prediction. In some embodiments, optionally, the localization image contains additional channels with other navigational features, similar to the channels listed above or of other nature. In some embodiments, optionally, the results of the training processes previously performed are used to decide which data of the input channels will be used based on its contribution to the success of the NavNN in predicting the outputs.

In some embodiments, in order to decide on the navigational driving action, the digital/virtual 3D localization image is inputted into the NavNN module, which can consist for example of a 3D Convolutional Neural Network (3D CNN). In some embodiments, the NavNN module processes the localization image in a “deep” multilayer scheme until outputting a probability per each possible driving action, for example using multiple sigmoid activation functions in its output layer. In some embodiments, a high-level module then selects the driving action with the highest output probability as the choice for the next navigational driving action, mechanically performing the driving action using automated motors or displaying the suggested driving action to the physician, as explained above. In some embodiments, the high-level module may also filter and/or improve and/or refine the outputs of the NavNN module. In some embodiments, for example, if the maximal output probability is not much better than the rest, then the high-level module may randomly choose between the two comparable outputs in order to introduce some beneficial randomness (exploration) into the system. In some embodiments, a potential advantage of this randomness is that is potentially helps evading local extremum points of the navigational system, where the system may go back and forth about the same point in space. In some embodiments, alternatively, the high-level module may force some hysteresis on the output probabilities so as to avoid fast transitions between different driving actions, thus smoothing the driving process.

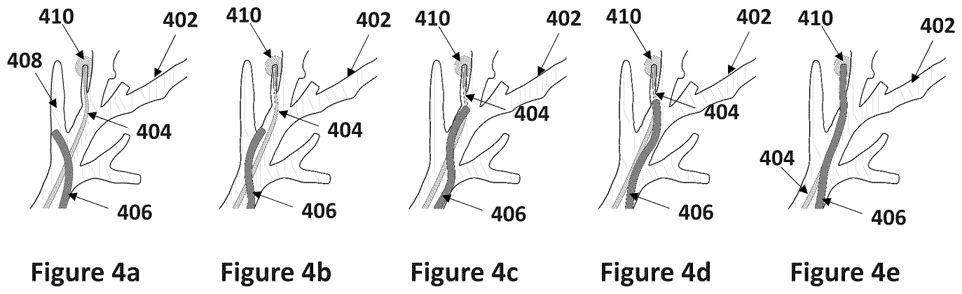

Referring now to FIGS. 4a-e, showing schematic representations of exemplary sequence of driving actions based on real-time localization images, as generated in real-time during procedure and processed by the NavNN module, according to some embodiments of the invention. In some embodiments, the output driving actions are optionally performed by automated motors. For the explanations of FIGS. 4a-e, it is assumed that the catheter is a passive “J” catheter and the driving system is a 2-actions system: ROLL and PUSH. Additionally, the luminal structure is marked as 402, the pathway to the target is marked as 404 and the catheter is marked as 406. FIG. 4a shows that the catheter 406 points left to an airway 408 which does not lead to the target 410, as indicated by a sphere at the end of a pathway. In some embodiments, the NavNN module processes the localization image and outputs the highest probability for a ROLL action. In some embodiments, the high-level module performs a motorized action to roll the catheter which results in a catheter as shown in FIG. 4b. In some embodiments, presented with localization image as shown in FIG. 4b, the NavNN module now outputs its highest probability for a PUSH action, which results in the image as shown in FIG. 4c. In some embodiments, the NavNN module then outputs ROLL again leading to the image as shown In FIG. 4d, where the catheter points at the target. In some embodiments, it only remains to push the catheter down the small left airway towards the target, as indicated by a PUSH output from the NavNN module, which results in final state as shown in FIG. 4e where the target is reached.

In some embodiments, the NavNN module produces real-time navigational instructions. In some embodiments, as mentioned above, the 3D localization image is a multi-channel volumetric image containing important navigational features (although it may also be a 2D view, as mentioned above). In some embodiments, while some of the features may be considered static (for example, the segmented lumen structure), others may and/or can change rapidly during procedure. For example, the fully tracked catheter position changes rapidly in real-time, which requires the 3D localization image to be updated accordingly. Moreover, in some embodiments, while the segmented lumen structure might be considered as static, it is much preferable to use a dynamic structure, for example, one which approximates the true deformed state of the lumen structure (or at least the virtually calculated deformed state of the lumen structure) during procedure. In some embodiments, hypothetical real-time deformation is tracked, or virtually calculated, during procedure (for example, using skeletal-based models or using a Deformation Neural Network as will be explained below, and also as further explained in International Patent Application N. PCT/IL2021/051475, the contents are incorporated herein by reference entirely), which causes the lumen structure to be modified according to the tracked/virtually calculated deformation and the localization image is updated accordingly to reflect the hypothetical real-time deformed state of the lumen.

In some embodiments, one or more techniques are used for generating a real-time 3D volumetric image based on known structures. In some embodiments, the lumen structure and the pathway to target may be static and generated once, while the fully tracked catheter is live and drawn on top of the static lumen map and pathway using 3D line rasterization techniques; all that while ignoring deformation. In some embodiments, the lumen structure and pathway to target may be dynamically modified, approximating the real-time deformed state of the lumen structure. In some embodiments, in this scenario, these features are updated in real-time which optionally requires a more computation extensive technique. In some embodiments, a novel approach is to use a GPU for rendering the 3D localization image in real-time. In some embodiments, in this setting, the 3D localization image is bound as a 3D render target and each of the navigational structures is rendered by breaking it into a set of volumetric pyramids. In some embodiments, in this novel proposed setting, instead of using planar triangles, the 3D volumetric features, such as the lumen structure, are volumetrically “tessellated” using 3D pyramid primitives. In some embodiments, an optimized GPU algorithm then processes the set of pyramids in a manner similar to the processing of standard 3D surface triangles and rasterizes them onto the 3D render target, essentially filling all the voxels inside the pyramids until the entire 3D volumetric structure is drawn in voxels. In some embodiments, while modem GPU hardware does not support rendering of pyramid primitives into a 3D render target as mentioned above, it can be extended to do so using dedicated GPU programs, for example by implementing an optimized GPU 3D rasterization algorithm using NVIDIA's CUDA (Compute Unified Device Architecture) or OpenCL (Open Computing Language). In some embodiments, alternatively, a dedicated GPU hardware can be used for rendering the 3D primitives, implemented in ASIC or FPGA. In some embodiments, the rasterization of 3D volumetric primitives (pyramids) onto a 3D render target can be done efficiently as the rendering of 3D surface primitives (triangles) onto a 2D render target, for example, using bucket rendering techniques in a parallel computing setting, as can be implemented for example in CUDA/OpenCL or in ASIC/FPGA. In some embodiments, the developer can then access the added features using an OpenGL extension or with DirectX. For example, when using OpenGL, instead of creating a 2D frame buffer, the developer will be able to generate and bind a 3D frame buffer for a GL_TEXTURE_3D render target, and instead of drawing primitives of type GL_TRIANGLES, the developer would draw primitives of type GL_PYRAMIDS (a new GLenum type) consisting of 4 vertices per primitive. When using DirectX, the developer will be able to create and bind 3D render target texture with the D3D11_BIND_RENDER_TARGET bind flag, and instead of drawing primitives of topology D3D_PRIMITIVE_TOPOLOGY_TRIANGLELIST, the developer would draw primitives of topology D3D_PRIMITIVE_TOPOLOGY_PYRAMIDLIST, consisting of 4 vertices per primitive.

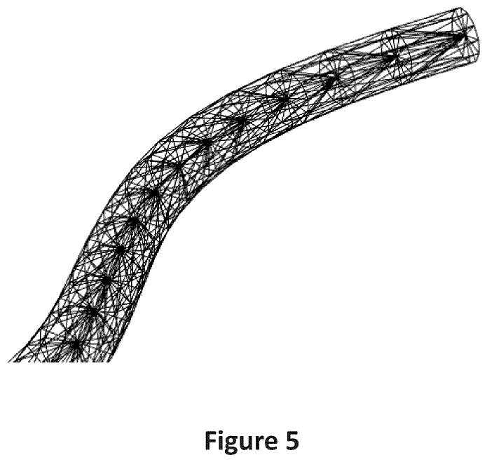

Referring now to FIG. 5, showing a schematic representation of an exemplary volumetric tessellation of a catheter using 3D pyramid primitives, according to some embodiments of the invention. In some embodiments, representing the 3D structures (for example: lumen structure, pathway to target and the fully tracked catheter —exemplary catheter shown in FIG. 5) using 3D pyramid tessellation provides great flexibility for moving and deforming them in real-time, thus reduces the complexity of generating a real-time 3D composite localization image for the NavNN module which is deformation aware. In some embodiments, to update the localization image, it is only needed to update the vertices which constitute the navigational features, for example, the catheter vertices are updated according to the fully tracked catheter position, as reported by the tracking system, the lumen structure and the pathway to target are potentially updated according to a real-time deformation tracking system, by updating their vertices according to their association to the original lumen segmentation or skeleton.

It should be noted that the method described above can be viewed as a general method for generating real-time 3D composite data using a dedicated GPU program or ASIC/FPGA, to be processed by a 3D Neural Network, for general use. For example, in some embodiments, the method can be used for rendering a real-time composite volumetric image of cars driving on a road for autonomous driving or for the real-time prediction of potential car crashes. As another example, the method can be used for the real-time rendering of a human's hand and fingers, as may be tracked by a plurality of sensors, to a 3D volumetric image. The 3D composite image can then be processed by NN for real-time gesture recognition or any other suitable use.

In some embodiments, the NavNN module is trained using several supervised and unsupervised methods. In some embodiments, when supervised, a realistic navigation simulator module is utilized. In some embodiments, the module may model the catheter using finite elements and may use Position Based Dynamics to simulate the physics of the catheter and to handle collisions between the catheter and the lumen structure. In some embodiments, the lumen structure may be represented using its skeletal model or using its raw segmentation volume, as was segmented from a CT scan (or MRI scan, or angiogram, etc.). In some embodiments, a distance transform may be applied to the segmented luminal volume and can be processed to create a 3D gradient field of the luminal structure in 3D space, simplifying collision detection between the simulated catheter and the luminal structure. In some embodiments, the catheter tip and/or curve can be presented inside the luminal structure using navigational views, for example, as done in real Navigational Bronchoscopy procedures. In some embodiments, an operator may then navigate the simulated catheter using a keyboard, a remote controller or any suitable method inside the lumen structure towards an arbitrarily selected target. In some embodiments, recordings of simulated navigations may then be collected. In some embodiments, in each timestamp the simulated state of the full catheter inside the luminal structure is completely known by the simulator. In some embodiments, the simulator module may generate a localization image as described above of the catheter inside the luminal structure along with a pathway to target based on the known simulated states. In some embodiments, in case a camera channel should be included, a virtual camera image can be rendered using ray tracing techniques, which resembles actual camera images for a specific camera specification (for example, as done in virtual bronchoscopy). In some embodiments, the camera image may be used as a 2D frame without depth information, or a depth channel may be included and can be computed by the simulator. In some embodiments, the localization image may then be associated with the operator's driving instructions. In some embodiments, the operator's instructions are therefore considered as labels for the NavNN module per each generated localization image in time. In some embodiments, the plurality of collected localization images along with their supervised labels (the operator's instructions) are then used in a supervised training process for the NavNN module. In some embodiments, the result of the training process is that the NavNN module tries to imitate the operator's instructions. In some embodiments, in the worst case scenario, the system just imitates the “average” operator's decisions and in the best case scenario, the system provides additional generalization on top of the operator's instructions. In some embodiments, the simulator module may be given to multiple operators and each operator may navigate to multiple different targets inside the simulated lumen structure of multiple patients. In some embodiments, during this process, a huge quantity of labeled samples is generated for the training of the NavNN nodule, which makes the training more robust and error tolerant.

In some embodiments, instead of using a simulator module, labeled training samples may be collected from actual navigational procedures performed on real patients and/or on mechanical simulated models, such as a plastic or silicon model of a luminal structure and/or for example preserved lungs, inflated in a vacuum chamber. In some embodiments, the navigational procedure may be “robotic” in the sense that the operator drives the system with a remote control, instructing the driving mechanism to perform any of several possible driving actions (for example, PUSH/PULL, ROLL, DEFLECT). In some embodiments, in the robotic case, labeled training samples are gathered by associating each real-time generated localization image with the operator's robotic instruction (e.g. PUSH/ROLL/DEFLECT). In some embodiments, a potential advantage of using data from real navigational procedures is that the catheter physics are realistic, whereas in the simulated case the catheter physics is only an approximation of reality. In some embodiments, the labeled localization images may be gathered from a plurality of procedures, performed using different systems on many patients. In some embodiments, the data collection does not interfere with normal procedure, since it is done in the background and may even be done offline in a procedure post-processing stage. In some embodiments, the procedure software may only record the data and states of the system over time (e.g., full catheter positions, selected target, deformation state of the lumen structure, camera video and robotic driving actions). In some embodiments, the post-processing stage then generates the corresponding localization images based on the recorded system states and labels them with the recorded robotic driving actions of the same timestamps. In some embodiments, labeled localization images can then be sent back to a dedicated server over local network or the internet or can be manually collected by field technicians. In some embodiments, the gathered data is used for training from scratch or improving the training of the NavNN module. In some embodiments, the NavNN module then imitates the driving actions and the navigational decisions of multiple physicians, which can potentially make it as good as or even superior to the most skilled physicians.

In some embodiments, when the navigational procedure is fully or semi manual (i.e., the catheter is handheld and is manipulated manually by the physician, without the help of a full driving system) it may be more difficult to label the localization images based on the manual manipulations of the catheter. In some embodiments, with manual operation, the manipulation of the catheter is not well defined as a choice from a set of several driving actions, as with the robotic system, but rather is the result of the physician's hand, wrist and arm manipulations. In some embodiments, in this case, a label can still be associated with each localization image by classifying each manual maneuver into a limited set of driving actions as mentioned above. For example, the most proximal tracked sensor of the fully tracked catheter (the one which is closest to the catheter's handle) may be used to classify the momentary handle maneuver, since it most efficiently reflects the operation which is done to the catheter's handle (as the robot would've done). As an example, when the physician pushes the catheter forward into the lumen structure the most proximal tracked sensor is most likely to be pushed forward, thus classifying the momentary maneuver as a PUSH action. On the contrary, the most distal catheter sensor (at the catheter's tip) might not move at all, for example due to frictional forces, which demonstrates why the proximal part of the catheter is much preferable in identifying the nature of the manual handle maneuver. As another example, when the physician rotates the handle, the proximal sensor is most likely to rotate in accordance with the catheter's handle, identifying the momentary maneuver as a ROLL action, while the distal sensor, again, might stay in place. In some embodiments, the catheter handle may be tracked using a dedicated sensor in the handle (for example, a 6-DOF tracked sensor, an IMU sensor (accelerometer, gyroscope, magnetometer or any combination), or any other suitable sensor). In some embodiments, in the case of a single or multi-joints deflectable catheter, the distal sensors may be used in order to detect the deflection of the catheter and provide the proper labeling for the NavNN module. In some embodiments, alternatively, since the deflection of the catheter's tip is done by the pushing and pulling of steering wires inside the catheter handle, special sensors can be placed in the handle to track the state of the steering wires and detect DEFLECT actions for the NavNN module. In some embodiments, is it therefore possible to collect recordings of manually operated catheters in real navigational procedures and label them in a postprocess stage, by classifying each momentary maneuver of the catheter handle, for example, using the most proximal tracked part of the catheter into a limited set of driving actions, as required by the NavNN module training.

In some embodiments, instead of using the above supervised training methods, the software simulator may also be used for unsupervised training using reinforcement learning. In some embodiments, in this case the NavNN module has full control on the simulated catheter and its goal is to drive it to a randomly picked destination target in a random patient simulation. In some embodiments, the NavNN module is rewarded whenever it makes notable progress down the pathway towards the target and is punished when it makes ineffective moves. In some embodiments, the goal of the training is to maximize the total reward of the NavNN module. In some embodiments, a potential advantage of unsupervised training such as this is that the NavNN module can be trained in parallel over thousands of simulations of different patients and targets without requiring human operators.

In some embodiments, the system is provided with dedicated commands (instructions) that allow for a level randomness or “exploration” to the navigation. In some embodiments, a potential advantage of providing the system with such apparent liberties is that it potentially avoids the risk of getting caught in a local probability extremum point, for example, where the NavNN module endlessly outputs PUSH/PULL actions back and forth about the same anatomical point, which leads to a navigational “dead-end” from which the NavNN module is unable to escape, when using, for example, a stateless Neural Network (i.e., one without “memory”) such as a 3D CNN on a single localization image input. In some embodiments, this certain level of randomness (or “exploration”) may be introduced into the navigation, for example by the high-level operating module. In some embodiments, the high-level operating module may prefer a random driving action at a certain probability over actions outputted from the NavNN module. In some embodiments, the high-level operating module may also detect “loops” (situations where the NavNN module oscillates about a local probability extremum point) and kick the NavNN module out of a loop by forcing random exploration. For example, the high-level module may force the driving mechanism to do a 100 ms ROLL action every second. In some embodiments, this action is harmless to the navigation process and may allow the NavNN module to escape from a local extremum point when it falls into one.

In some embodiments, the NavNN module utilizes previous recorded states of the catheter. In this case, the NavNN module is no longer perfectly “momentary”. Instead, in some embodiments, the NavNN module bases its output on history and not just on the current localization image input. In some embodiments, the NavNN module is therefore trained on time sequences of localization images instead of training on randomly shuffled single localization images. In some embodiments, the NavNN module is then inputted a localization image as before, together with the output state of the previous prediction, and outputs an updated state for the next prediction. In some embodiments, the NavNN module is equipped with memory that allows the NavNN module to “remember” that it already tried a certain maneuver and “see” that it didn't succeed, thus escaping loops by trying different techniques instead of repeatedly trying the same maneuver. In some embodiments, in a more general setting, the NavNN module is inputted a short sequence (for example containing 30 last frames) of past localization images and their output actions together with the current one thus basing its output on history without using a dedicated state vector. In some embodiments, the NavNN module may be implemented using 3D CNN over a short sequence of past localization images, or using a 3D Recurring Neural Network (3D RNN) with state vectors or by any other suitable methods, with or without memory.

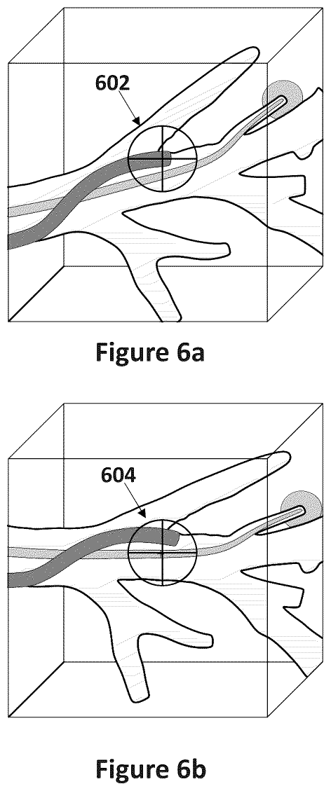

Referring now to FIGS. 6a-b, showing a schematic representation of exemplary 3D localization images centered according to different objects, according to some embodiments of the invention. In some embodiments, since the NavNN module is given an image (the localization image) without being told where the catheter is located inside this image or in which direction the catheter points, the NavNN module might then be forced to search for the catheter inside the image, which is a useless effort since the information about the catheter's full position is already known to the high-level module. In some embodiments, the task of the NavNN module is “eased” by providing it with an input image in which for example the catheter's tip is centered 602 and the image's X-axis is aligned with the catheter's tip direction, as shown for example in FIG. 6a. In some embodiments, the NavNN module can then learn that the catheter is always located at the center of the image and points towards the X-axis and focus on the rest of the navigational features to decide on the best driving actions. In some embodiments, the localization image may be centered 604 and oriented according to the closest point along the pathway to target relative to the catheter's tip, as shown for example in FIG. 6b. In some embodiments, it may be oriented such that the image's X-axis is aligned with the pathway direction to the target and the image's Z-axis may be aligned with the normal vector of the next bifurcation, or with an interpolated normal vector between last and next bifurcations. In this scenario the localization image maintains a rather stable center and orientation along the pathway to target regardless of catheter's tip maneuvers, since it's no longer bound to the catheter's tip but instead it is tied to the pathway to the target. In some embodiments, several other options for centering and orienting the localization image can be used which may be combinations of the options mentioned above. For example, the localization image may be centered at the catheter's tip but oriented according to the pathway to target, or vice versa. In some embodiments, additionally, the size of the localization image can be increased or decreased and the resolution can be changed as well. In some embodiments, any such configuration among others can be used for training and prediction in the NavNN module.

### Exemplary Deformation Neural Network (DeformNN) Module

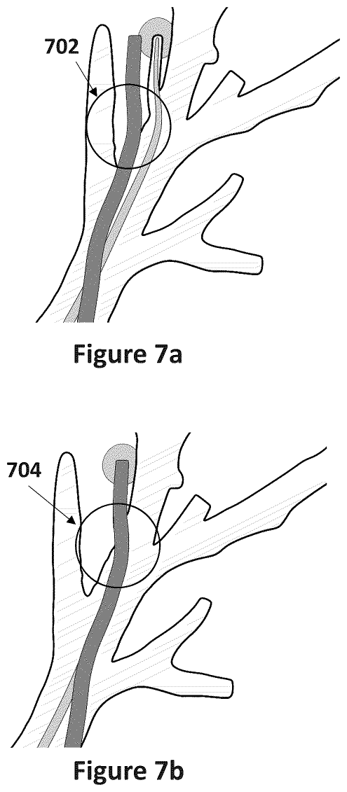

Referring now to FIGS. 7a-b, showing schematic representations of exemplary non-deformed and deformed localization images, according to some embodiments of the invention. In some embodiments, as mentioned above, accurate real-time localization images of the device are provided to the NavNN module to potentially produce better driving actions. In some embodiments, the localization image contains, in addition to the luminal map and in a separate channel, the fully tracked catheter on top of the lumen structure. In some embodiments, to provide more information and increase the accuracy of the DeformNN, the localization image contains additional channels of additional tracked catheters. In some embodiments, in order to increase the accuracy of the NavNN module and/or for allowing the catheter to be located in its correct position inside the anatomy, a deformation input is provided to the NavNN module, which comprises information regarding real-time based information on the actual organ deformation, which is translated to deformations in the lumen structure as shown in the localization image. In some embodiments, this is accomplished by deploying a skeletal model of the lumen structure and used for finding the organ deformation based on the fully tracked catheter using optimization methods, which were also further explained in International Patent Application N. PCT/IL2021/051475, the contents are incorporated herein by reference entirely. In some embodiments, as shown for example in FIG. 7a, with a non-deformed lumen structure (one that doesn't possess any deformation compensation), a non-deformed image may be constructed. In FIG. 7a the catheter may seem to cross 702 the boundaries of the lumen. In some embodiments, a downside of feeding the NavNN module with a non-deformed localization image is that the performance of the NavNN module will be potentially degraded since it's not provided with an accurate image of the catheter inside the lumen. In some embodiments, the deformation tracking algorithm provides either adjustments in the catheter's position relative to the lumen structure or vice versa such that the catheter will appear inside the allowable tubes, as it does in reality. In some embodiments, in a skeletal model-based deformation tracking algorithm, the lumen structure is modeled as a skeleton with branches of certain radius and connecting bifurcations. In some embodiments, the skeleton is deformed according to certain deformation models so as to bring the catheter back inside the lumen under imposed organ shape constraints.

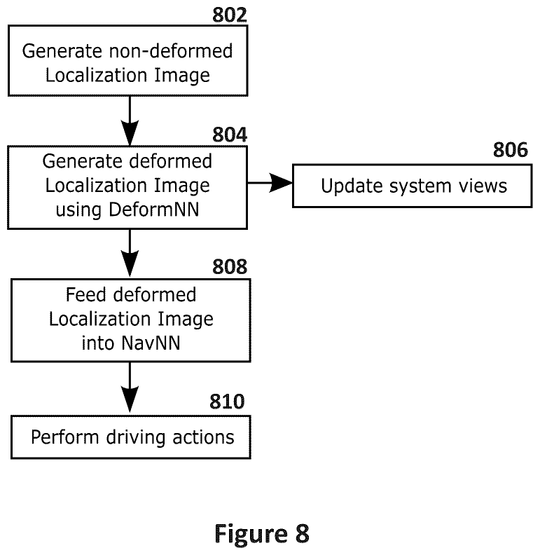

In some embodiments, a new method is proposed for finding the lumen deformation based on an AI statistic approach. In some embodiments, instead of explicitly modeling the lumen structure with a skeletal model and finding the deformation based on optimization methods, an Al approach is followed in which the deformation is solved implicitly using a Neural Network. In some embodiments, similar to the NavNN module, the DeformNN module is inputted with a localization image which can be of the same size and/or centered and/or oriented, as discussed above. In some embodiments, however, the DeformNN module is not necessarily inputted with the pathway to the target as one of its input channels, since this information is more relevant for navigating to a target but less relevant for finding the lumen deformation. In some embodiments, in addition, while the NavNN module is preferably inputted with a deformed localization image (which possess deformation compensation), the input to the DeformNN module is a non-deformed localization image, as shown for example in FIG. 7a. In some embodiments, the localization image inputted to the DeformNN module can further contain a camera channel, as shown for example in FIG. 3b. In some embodiments, the DeformNN module utilizes the camera channel for deciding on the most probable deformation of the lumen structure. For example, when the lumen structure is deformed, as shown for example in FIG. 7a, the camera image may teach on the correct catheter position inside the anatomy, for example, since it localizes the catheter's tip relative to visual bifurcations. In some embodiments, the DeformNN module may learn to use these features for better finding the correct anatomical position of the catheter in the deformed lumen structure. In some embodiments, the DeformNN module is responsible for taking a non-deformed localization image (lumen structure and catheter position) and transforming it into an accurate deformed localization image of the same size, as shown for example in FIG. 7b. In some embodiments, this can be achieved for example using a 3D U-Net Neural Network architecture. In some embodiments, the output deformed localization image can then be rigged with the additional channels (pathway to target with the applied deformation) and inputted to the NavNN module to produce a more reliable driving action, leading the catheter accurately towards the target. In some embodiments, the output of the DeformNN module may also be used for display, to correct the 2D/3D system views to reflect the lumen deformation, as will be further explained below and show for example in the flowchart in FIG. 8.

Referring now to FIG. 8, showing a flowchart of an exemplary method of displaying correct 2D/3D system views to reflect the lumen deformation, according to some embodiments of the invention. In some embodiments, the system generates a non-deformed localization image 802. The term “non-deformed localization image” refers to a localization image where the image has not been altered and/or compensated for potential and/or estimated and/or calculated deformations (either due to the movement of the catheter, or the movements of the patient, etc.). In some embodiments, the system then generates a deformed localization image using the DeformNN module 804. The term “deformed localization image” refers to a localization image where the image has been altered and/or compensated for potential and/or estimated and/or calculated deformations (either due to the movement of the catheter, or the movements of the patient, etc.). In some embodiments, the system views are updated with the newly generated deformed localization images 806. In some embodiments, the newly generated deformed localization images are then fed into the NavNN module 808. In some embodiments, then the NavNN module provides the necessary driving actions, which will be performed by the system 810.

In some embodiments, instead of outputting a deformed version of the non-deformed localization image input, the DeformNN module may only output one or more probabilities, indicative of the input catheter to be within the lumen in its correct position, as in the input localization image. In this scenario a high-level optimization is used, for example, one that is based on a skeletal model, and the deformation of the lumen is searched as with deformation tracking algorithms that are based on the skeleton approach. In some embodiments, when outputting a single probability, instead of basing the optimization on the energy minimization of more standard energy functions (such as ones that encode bifurcation angle constraints etc.), the optimization is done so as to maximize the output probability of the DeformNN module —a deformation state is searched such that the probability of it being the correct one, as outputted by the DeformNN module, will be maximized. In some embodiments, the DeformNN then serves as a metric for evaluating a proposed deformation, but the deformation itself is done externally in an optimization algorithm using any suitable deformation model.

In some embodiments, the DeformNN module may be designed and trained to output the catheter's position in a fully deformed localization image, as described above. In some embodiments, the DeformNN module takes an input catheter position on a non-deformed lumen structure and renders it on its output inside the non-deformed lumen structure, where it should have been had the lumen structure was not deformed. In some embodiments, the DeformNN module outputs a single channel on which it renders the modified catheter position. This is different to what was shown in FIGS. 7a-b, in which the DeformNN module modifies the lumen structure from a non-deformed to a deformed state, but leaves the catheter intact. In this case the high-level module may find the catheter in the output image and match between the input catheter in its original position and the output catheter, in its deformed position inside the anatomy, as outputted by DeformNN module. In some embodiments, the catheter matching can be achieved by finding the catheter's tip and climbing up along the catheter's length in both 3D images, or by any other suitable method. In some embodiments, the catheter's position before and after deformation can be represented using curve functions

γ₀, γ₁: [0,1]→R³
respectively. In some embodiments, a set of deformation differences can then be computed using

Δγ=γ0−γ1

In some embodiments, since the DeformNN module finds the catheter's position inside the anatomy, it can be safely assumed that γ₁(σ) resides inside the lumen structure. In some embodiments, each 3D position along the non-deformed lumen structure γ4(σ) can then be updated to its deformed position γ₀(σ) using any suitable skeletal model for display or other computation algorithms. In some embodiments, by matching the catheter positions before and after deformation (as outputted by DeformNN module) the deformation of the lumen structure is revealed indirectly.

In some embodiments, alternatively, in a more direct approach, the DeformNN module may be designed and trained to output the deformed lumen structure based on the catheter position, leaving the catheter intact. In this case, the output image is the deformed version of the input luminal structure and it can be used for display or other computation algorithms. For example, the output luminal structure can be matched to the input luminal structure using 3D image registration techniques or by using the skeletons of each the input and output structures. In some embodiments, by matching between the input and output structures, the deformation vectors can be computed for each shared point inside the input and output structures. In some embodiments, the deformation vectors can then be applied on a skeletal model of the luminal structure to bring it to track its deformed state as solved by DeformNN module in real-time.

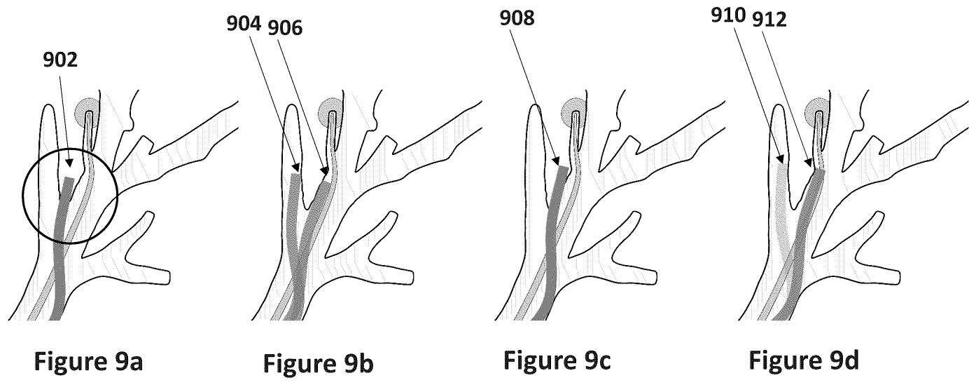

Referring now to FIG. 9a-d, showing schematic representations of exemplary actions performed by the DeformNN module, according to some embodiments of the invention. In some embodiments, in certain cases where it is difficult to determine whether the catheter is inside one lumen or another 902 due to high symmetry or severe preregistration system errors, as shown for example in FIG. 9a, the DeformNN module, if designed to render the catheter in its modified position inside the anatomy, may choose to output two possible hypothetical catheters 904, 906 with similar or different intensities (probabilities), as shown for example in FIG. 9b. In some embodiments, this indicates that the DeformNN module is uncertain of the correct deformation, and each output catheter's intensity reflects the AI's confidence of the particular position. In this case, the high-level module may pick one of the output catheter curves based on the output intensities or based on other high-level considerations. For example, the high-level module may choose to display the catheter which is closer to the catheter already presented by the system, thus preventing “jumps” between different catheter hypotheses (especially in cases where the output intensities are similar). In some embodiments, alternatively, the split catheter may be displayed to the user as to reflect the system's uncertainty of the actual catheter position inside the anatomy. In this view the operator is presented with two or more hypothetical catheters inside the lumen structure, each being displayed with a different intensity or opacity which corresponds to its output intensity by the AI In some embodiments, the user can have this information for “informative” purposes alone. In some embodiments, the user can use this information to tell the system which direction to take. In some embodiments, once the ambiguity is resolved, for example, after advancing the catheter 908 farther towards the target so that its curve is of more definitive shape, which teaches the DeformNN module about the actual catheter position inside the anatomy, as shown for example in FIG. 9c, the split catheter output intensity diminishes naturally 910 and the DeformNN module outputs a single strong catheter intensity 912 at its outputs, as shown for example in FIG. 9d. Accordingly, in some embodiments, the system views eventually show a single strong catheter at a resolved position inside the anatomy as all other hypothetical catheters diminish in opacity once the ambiguity is resolved. In some embodiments, in an alternative view, when the DeformNN module outputs multiple catheter hypotheses, the system may choose to present the catheter only down to the point where it begins to split (as outputted by the DeformNN module). In some embodiments, it may then render the rest of the catheter (i.e., the left and right splits) in “red” or with transparency to indicate to the user that the system is uncertain about the position of this part of the catheter. In some embodiments, in another alternative view, upon catheter ambiguity, the screen may split, for example into a left and right screen, each displaying a different hypothetical position of the catheter inside the anatomy. In some embodiments, once ambiguity is resolved, the “winning” half grows into a full screen view, pushing the other half out of view. In some embodiments, the NavNN module can be presented with a localization image which contains multiple catheter hypotheses (with possibly different intensities) and can be trained such that it will still be able to continue navigation even under these ambiguous conditions. For example, if the NavNN module employs memory, it can try a certain driving action which leads into a conclusive catheter position. In some embodiments, the NavNN module may then “see” if the conclusive catheter position is advanced towards the target. If it isn't it may choose to pull the catheter back and try a different driving action (since it already tried the first driving action, as encoded in its memory or state vector), such that the final conclusive catheter position will advance towards the target.

In some embodiments, training the DeformNN module is done by presenting pairs of non-deformed input and deformed output localization images. In some embodiments, these images can be collected by using a realistic simulator module, as described above for the training process of the NavNN module. In this scenario the catheter's exact simulated position is known to the simulation. In some embodiments, the catheter's true position in simulation inside the lumen structure is used to generate the output localization image for the DeformNN module. In this image, the catheter is placed exactly at its true position inside the anatomy, as should be outputted by the DeformNN module. In some embodiments, to create the input images, some deformation model is applied to the lumen structure. For example, the structure can be deformed randomly based on standard polynomial or spline techniques or using more elaborate techniques which imitate the anatomical deformation of true organs, for example, using a finite elements and/or finite volume physical simulation which may be based on physical measurements of various tissues and structures. In some embodiments, since the deformation is only applied to the luminal structure, but not to the catheter, the result is a “non-deformed” localization image (one which doesn't possess deformation compensation) in which the catheter may seem to cross lumen boundaries. In some embodiments, this creates a pair of images which can be used for the training of the DeformNN module. In some embodiments, similarly to NavNN module, collecting data from simulation has the potential of creating a large set of training samples over many patients, targets and different catheter poses inside the lumen structure which is important for successful training of the AI model.

In some embodiments, recordings of true procedures may be used to collect accurate deformation data of live organs, for example of the lungs. In some embodiments, the catheter may be introduced to certain known airways inside the lungs and the catheter's full position can be recorded under certain forced or natural deformations, which teaches about the deformation of that airway. In some embodiments, additionally, multiple catheters may be introduced to multiple known airways and their full positions can be recorded to teach about the deformation of a plurality of airways in parallel under certain applied forces. In some embodiments, training samples can also be collected from a mechanical simulated model, as with NavNN module. In some embodiments, a plurality of tracked sensors can be deployed inside the organ, for example, on the pleura of the lungs, and can record real-time data of deformation. In some embodiments, multiple CBCT (Cone-beam CT) scans can be performed while deforming the organ, and the different scans can be registered using deformable registration to reveal the deformation vectors between the scans under certain applied forces. In some embodiments, the deformation can be learned and measured by other means as well, for example, using ultrasound probe, fluoroscopic imaging, by use of contrast, markers, extracorporeal sensors among other suitable means.

In some embodiments, the DeformNN module can be further trained in a pre-procedure stage on the specific patient using deformation augmentation methods as described above, to further fit the model on the specific patient's lumen structure, thus increase the AI model's performance during procedure. For example, prior to procedure, the patient's lumen structure can be loaded into an offline simulator module. In some embodiments, a simulated catheter may then be placed in different random locations inside the simulated lumen structure. In some embodiments, deformations of the lumen structure can be simulated by the simulator module, creating pairs of non-deformed vs. deformed localization images. In some embodiments, a trained DeformNN module can be presented with the newly created image pairs and can be further trained based on these pairs with small learning rate, such that it will still possess its weights from its original training, but these weights will now be fine-tuned towards fitting deformations of the present patient. In some embodiments, these actions potentially tweak and bias the deformation model onto the current patient, slightly losing its generality in favor of performance for solving deformation on the current patient's anatomy.

Exemplary Stress Neural Network (StressNN) module

In some embodiments, the system comprises a Catheter Stress Detection algorithm, which utilizes the tracked catheter's position and shape in its anatomical position to estimate catheter stress inside the patient's airways. In some embodiments, the algorithm examines the catheter's shape and provides alerts such as in cases where the catheter is about to break or starts to apply excessive forces on the airways. In some embodiments, these alerts can be used for example to supervise robotic driving maneuvers as well as provide alerts in the handheld case, for patient safety and system stability. In some embodiments, the algorithm is based on pure geometrical considerations as well as a dedicated Stress Neural Network (StressNN) module, which analyzes the shape of the catheter.

In some embodiments, while force sensors may be integrated inside the driving mechanism to predict the forces applied by the catheter to the airways (as done by a physician with a handheld catheter), another option is to utilize catheter tracking information relative to a robotic catheter advancing distance for estimating the catheter's stress inside the airways. In some embodiments, as described elsewhere herein, a catheter's fully tracked curve is analyzed, in its localized state inside the anatomy, to accurately predict the level of stress of the catheter inside the airways. Generally, when a catheter follows a smooth path, it is most likely relieved and will not harm the tissue. As the catheter starts to build a curvy shape inside straight airways, and as catheter shape loops are starting to form, the catheter's stress level is considered high and, when using a robotic driving mechanism, the robotic driving mechanism is stopped. In some embodiments, the catheter is then pulled and relieved. In some embodiments, a potential advantage of combining the proposed stress detection mechanism with external or internal force sensors is that it potentially provides a fuller protection for a robotically driven catheter.

In some embodiments, for example, when the catheter is driven a known distance forward, it is expected that the catheter's tip will advance accordingly. In the extreme case, where the catheter's tip doesn't move, it is concluded that tension was built along the length of the catheter and wasn't translated to forward motion of the tip. In some embodiments, additionally, it is possible to analyze the momentary shape of the catheter and deduce the stress level of the catheter length based on its shape inside the lumen structure.

In some embodiments, for example, a physical finite elements simulation which realistically simulates the physical properties of the catheter and the lumen structure can be used to estimate the forces applied by the catheter on the lumen structure for a given shape and position inside the anatomy. In this case, catheter is placed inside the simulated lumen structure exactly as it is located inside the realistic structure, as tracked in procedure. In some embodiments, these are performed in real-time during the intervention. In some embodiments, these are performed only in simulation, meaning not during a procedure, for example to teach the NN and/or other software. In some embodiments, the simulation is then played and physical simulated forces can be computed based on the catheter's simulated structure and the lumen's simulated behavior. In some embodiments, once the contact forces are computed, as well as the inner catheter forces, binary or smooth threshold may be used to compute a force risk estimate, for example, a scalar between 0 and 1.

In some embodiments, a 3D localization image, as the one described above, can be used to visualize the catheter's shape inside the lumen structure in 3D. In some embodiments, the localization image can be inputted into a dedicated Stress Neural Network (StressNN) module, which outputs a force risk estimate based on the catheter's shape inside the lumen structure, as visualized by the localization image. For example, the StressNN module may output a value close to 0 when the catheter is relieved inside the lumen structure and may output a value close to 1 when the catheter's shape inside the lumen structure indicates a risk (for example, when the catheter's shape is highly curvy or loops start to form). In this case the high-level module may pull the catheter back until the StressNN module outputs a value closer to 0 and the catheter is relieved. In some embodiments, in order to provide higher levels of reliability to the StressNN module a localization image of larger support is provided, for example, one in which the full catheter's trackable length is visible. In some embodiments, this allows the StressNN module to also take under consideration proximal parts of the catheter in which curves and loops may build during procedure.

In some embodiments, a simulator module is used to train the StressNN module. In this case, a simulated catheter is introduced into the lumen structure and navigates to random positions inside the organ. In some embodiments, contact forces and inner catheter forces are calculated by the physical simulation and labeled samples are gathered, by pairing localization images with their corresponding force risk estimates, based on the computed forces. In some embodiments, training of the StressNN module is performed by providing recordings of previous medical procedures, for example by using sensors in catheters during procedures and recording the forces. For example, by analyzing the recording and deriving actions performed by the user with the status of the catheter at that moment.

In some embodiments, the NavNN is also be used to detect catheter stress inside the luminal structure. In some embodiments, the NavNN is trained so that whenever the operator (or the simulator) detects a high level of catheter stress, the catheter is pulled back. In some embodiments, this teaches the NavNN to perform stress detection of the catheter as indicated in the 3D localization image, and to pull the catheter back in cases where stress is being built. In some embodiments, the final catheter stress detection is performed by a physical simulation module, a dedicated StressNN module, the NavNN module, or any combination of the above.

Summary of the Exemplary Endoluminal Device with Tracking and Navigational Systems Thereof

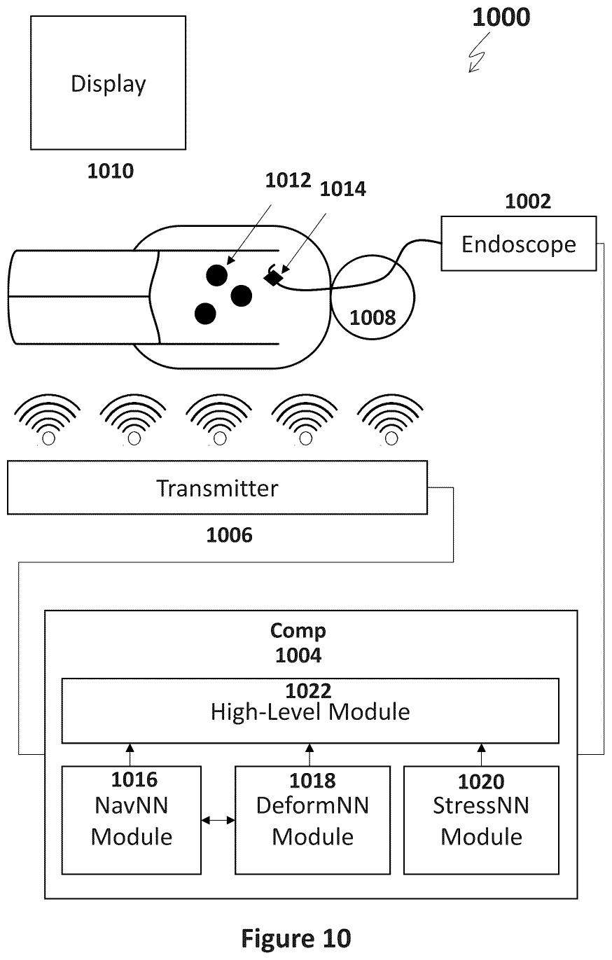

Referring now to FIG. 10, showing a schematic representation of an exemplary endoluminal device with the tracking and navigational system, according to some embodiments of the invention. The endoluminal device shown in FIG. 10 is a modified version of the endoluminal device shown in FIG. 1, with the additions of the components responsible for providing inputs regarding navigation, deformation and stress. In some embodiments, the endoluminal system 1000 comprises an endoluminal device 1002, for example an endoscope or a bronchoscope or a vascular catheter, or a vascular guidewire, configured for endoluminal interventions. In some embodiments, the endoluminal device 1002 comprises one or more cameras and/or one or more sensors 1014 at the distal end of the endoluminal device 1002. In some embodiments, the endoluminal device 1002 is connected to a computer 1004 configured to monitor and control actions performed by the endoluminal device 1002, including, in some embodiments, self-steering actions of the endoluminal device 1002, as will be further explained below. In some embodiments, the endoluminal system 1000 further comprises a transmitter 1006 configured to generate electromagnetic fields used by the endoluminal system 1000 to monitor the position of the endoluminal device 1002 inside the patient 1008. In some embodiments, the endoluminal system 1000 further comprises a display unit 1010 configured to show dedicated images to the operator, which potentially assist the operator during the navigation of the endoluminal device 1002 during the endoluminal interventions. In some embodiments, the endoluminal system 1000 optionally further comprises one or more sensors 1012 configured to monitor movements of the patient 1008 during the endoluminal intervention. In some embodiments, the patient's movements are used to assist in the navigation of the endoluminal device 1002 inside the patient 1008. In some embodiments, the computer 1004 comprises a NavNN module 1016, configured to receive accurate real-time localization images, for example from the one or more camera and/or the one or more sensors 1014 and as explained above. In some embodiments, as mentioned above the NavNN module 1016 produces then driving directions for the endoluminal device 1002 inside the patient 1008 to reach a desired location therein. In some embodiments, the computer 1004 comprises a DeformNN module 1018, configured to calculate deformation information and provide the deformation information to the system 2D/3D views to produce a more accurate image of the catheter location inside the anatomy as well as to the NavNN module, which then utilizes that deformation information to potentially increase the accuracy of the navigation and driving directions. In some embodiments, the computer 1004 comprises a StressNN module 1020, configured to calculate and/or estimate stress performed by the catheter on the tissues where the endoluminal device 1002 is being maneuvered. In some embodiments, the StressNN module 1020 performs the calculations/estimations based on the catheter's position and location inside the body of the patient 1008, optionally in real-time. In some embodiments, the computer 1004 comprises a High-level module 1022, which receives all the information from the localization systems (transmitters and sensors), the NavNN module, the DeformNN module and the StressNN module, and utilizes this information to actuate one or more mechanisms in the endoluminal system 1000, for example robotic mechanisms that actuate the distal end of the endoluminal device 1002 (steering —see below), robotic mechanism that actuate advancement and/or retrieval of the endoluminal device 1002, into and from the patient.

### Exemplary Self-Steering Endoluminal Device and Augmented Views

In some embodiments, the endoluminal device 1002 comprises a mechanical working distal end configured to be either manually or automatically actuated for directing and facilitating the endoluminal device 1002 towards the desired location inside the body of the patient 1008. In some embodiments, the instrument (endoluminal device 1002) is configured such that it may autonomously orient its working tip towards a special target, having a suitable spatially-aware algorithm (based for example on the information received from the NavNN module and/or the DeformNN module) and sensing capabilities. For example, in the context of endoluminal device navigation, the system allows for a self-steering device, in which the operator is moving the device distally or proximally, while the tip of the device is self-steering in accordance with its position in relation to a target. In some embodiments, such target might be, for example, a point on a pathway, towards which the tip of the device is configured to be pointed. In this example, in order to follow the pathway to a target, an operator might only be required to carefully push the device distally, while the tip is self-steering through the bifurcations of the luminal tree such that ultimately the device reaches its target. Further to this example, in some embodiments, a pre-operative plan in made on an external computer device, such as a laptop or a tablet or any other suitable device, in which the luminal structure is segmented and the target and pathway are identified. In some embodiments, the plan may then be transferred to the device via physical connection, radio, WiFi, Bluetooth, NFC (Near-field communication) or other transfer methods and protocols.

In some embodiments, the point in space of the self-steering tip might be a target in a moving volume, for example a breathing lung, or for example a target in the liver, or for example a target in soft vascularity, or for example a target in the digestive system, whereas the tip of a catheter is configured to orient towards this target without operator intervention.

In some embodiments, the endoluminal device 1002 may comprise a handle which is encasing the required electronic processors and control components, including the required algorithms, a power source, and the required electro-mechanical drive components. In some embodiments, the endoluminal device 1002 may be a disposable device, or a non-disposable device.

In some embodiments, the endoluminal device 1002 may be connected to external screens on which a representation of the lumen structure is displayed, along with an updating representation of the position of the instrument inside the lumen. In some embodiments, additionally or alternatively of a display of the position of the instrument, other means of feedback are provided to the operator to notify the state of the system. In some embodiments, such notifications may be, for example, a blinking green-light as long as the instrument is on-track to reach the target (for example, it is following the pathway); a steady green-light indication when the target was reached. In some embodiments, a steady red-light indication or a vibration feedback using a vibration motor in the handle when the target may not be reached in the current location and the catheter needs to be pulled back (for example, when the tip is past the target, or when the tip is down a wrong bifurcation). In some embodiments, in addition to the indications mentioned above, sound indications may be played by small speakers inside the catheter's handle, guiding the operator through the procedure. In some embodiments, additional indications and alert methods are not mentioned here but lay within the scope of this invention.

In some embodiments, the electro-mechanical drive components can consist of miniature motors inside the catheter's handle. In some embodiments, there can be a single miniature motor controlling the roll angle of a passive “J” catheter. In some embodiments, the NavNN module 1016 may output two driving actions: PUSH/PULL, ROLL. In some embodiments, when a ROLL action is required, the high-level module 1022 automatically activates the roll motor inside the catheter to perform the rotation of the catheter, so that the catheter always automatically aligns with the next bifurcation to the target. In some embodiments, when a PUSH action is required, a green LED on the catheter's handle may blink, indicating to the operator that the catheter is on track to the target and needs to be manually pushed. In some embodiments, when a PULL action is required a vibration feedback may be activated in the handle, for example using a vibration motor inside the handle, or a red LED may turn on or blink, indicating to the operator that the catheter went off track and needs to be retracted. In some embodiments, when a PUSH or PULL action is required, the high-level module 1022 activates the forward/back motor inside the catheter to perform the limited advancement or retraction of the catheter, so that the catheter is automatically advanced towards the target (or pulls back when the catheter enters a wrong lumen). In some embodiments, the dimension (size or length) of the movement (either forward or back) performed by the catheter is limited by the mechanical characteristics of motor (optionally located in the handle of the catheter). In some embodiments, that dimension (size or length) is fixed and known. In some embodiments, that dimension (size or length) is actively adjustable and known, for example by either exchanging motors or by modulating the force provided by the motor. In some embodiments, the system is configured to use the “known dimension of movement” to provide fine tuning to the navigation towards the target. In some embodiments, alternatively or additionally, the system is configured to use the “known dimension” for maintaining stability inside the anatomy, for example, when reaching a moving target, by actuating the device (activation forwards, activation backwards and deactivation), the system can maintain a certain position albeit the movement of the target, therefore maintaining stability inside the anatomy in relation to the target.

In order to understand the complexity of the task, the following example will be provided. Breathing causes a natural deformation of the tissues, for example in the lungs, the lower lobes of the lung can move/deform from about 2 cm to about 3 cm during breathing. In order to provide the most accurate “image” for the navigation, the system, using the DeformNN module, updates the luminal map in real-time according to the sensed movements of the patient, for example, caused by the breathing. In some embodiments, the movements are monitored, for example, using one or more sensors positioned on the patient and/or on the bed and/or on the operating table. In some embodiments, once movement is accurately incorporated in the dynamic luminal map, other deformations are also taken into consideration, for example, the deformation caused by actually actuating the device to the location, and actively incorporated into the dynamic luminal map. Therefore, at this moment, there is a 3D luminal map that is being constantly updated for deformations caused by organic or induced motion. Once this is achieved, the user can instruct the system to maintain a chosen position. For example, a given distance relative to a target (e.g. stay 15 mm from the target) or maintain position on a specific point on the luminal map chosen by the user so that the device is kept on the chosen point. In some embodiments, the system actuates the propulsion apparatus to accomplish the fine tuning of the navigation and positioning of the device using the system “awareness” of the “known dimension of movement” caused by the actuation. In some embodiments, a potential advantage of fixing the device to an anatomical location inside the luminal structure in relation to a moving target is that it is better to the alternatives of stabilizing a device in a free space, or stabilizing the device to luminal structure, which does not account for the movement of the actual target, which may have other movement characteristics from the lumen. A 3D tracking system usually tracks devices in tracking coordinates, which are usually relative to a transmitter (for example in EM), which is usually fixed to the bed. Devices are therefore tracked in “free 3D space”, that is, for example in bed coordinates. The device location may therefore oscillate significantly (for example between 2 and 3 cm) in its tracked x, y, z location due to patient's breathing or other organic or non-organic deformation, although the anatomical location of the device inside the body does not actually change, for example, the device is in the same location inside an lumen, but the target moves due to the deformation caused by the breathing. Known art usually fix a robotic catheter in free space by fixing the robotic device in the same x, y, z location “in free space” relative to the tracking source by applying some control mechanism on the catheter's location. In some instances, this method has a great downfall since a fixed x, y, z location relative to the source does not reflect a fixed location relative to the anatomy.

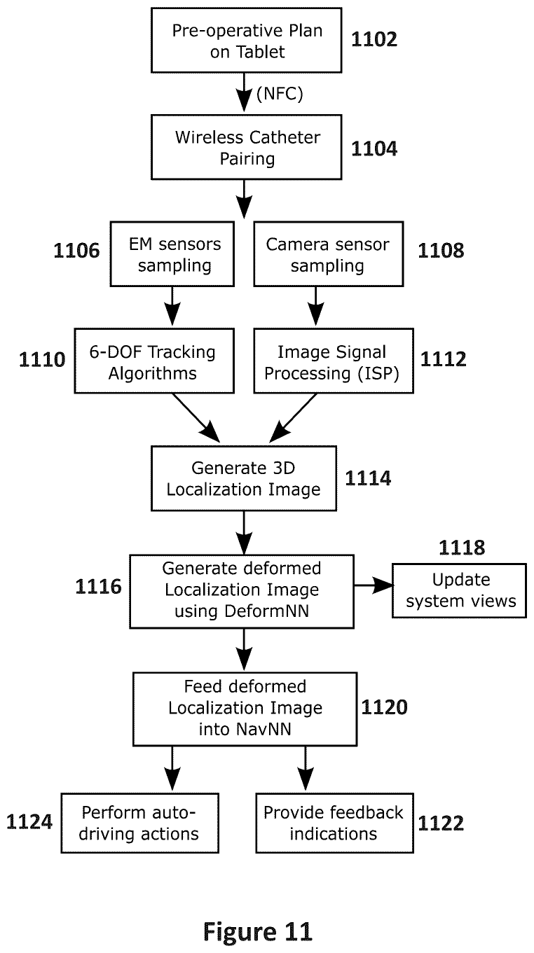

In some embodiments, the disposable catheter is completely wireless and contains a power source such as a battery, a microprocessor, a dedicated ASIC/FPGA, NFC communication support, a red/green indication LED, a vibration motor and a miniature rotation and/or forward motor for the catheter. An exemplary system flowchart is shown in FIG. 11. In some embodiments, a pre-operative plan is done on a tablet device for a specific patient and communicated to the wireless catheter using NFC by attaching the catheter to the tablet in a catheter-patient pairing stage 1102.

In some embodiments, optionally, a sound indication may be played or a LED may turn on upon pairing, that is, upon a successful transmission of the patient's plan onto the catheter 1104. In some embodiments, optionally, the plan may consist of a segmented luminal structure, a pathway plan to target and target marking. In some embodiments, optionally, the segmented luminal structure is of sparse nature and can therefore be compressed (for example, to just a few kilobytes) to fit the memory limitations of most microprocessors, for example, using Huffman Encoding or other suitable method. In some embodiments, optionally, an electromagnetic calibration may also be transferred to the wireless catheter upon pairing. In some embodiments, optionally, an electromagnetic transmitter identifier or full configuration and calibration may be transferred to the wireless catheter so that the catheter will be able to perform fully calibrated electromagnetic tracking during procedure. In some embodiments, camera sensor sampling is performed 1108. In the case that the catheter consists of digital electromagnetic sensors, no external amplifiers and DSP are needed to provide for full 6-DOF tracking, only software algorithms 1110 which can be implemented in most microprocessors (relying on the transferred electromagnetic configuration and calibration). In some embodiments, during procedure the catheter is then able to solve full catheter positions using 6-DOF tracking algorithms 1110 by processing the measured magnetic fields from its plurality of sensors, as previously explained. In some embodiments, the catheter positions are then matched to the luminal structure in one or more registration processes. In some embodiments, a multi-channel 3D localization image may be rendered 1114, optionally in real time, using methods mentioned above, for example using a special GPU block in the dedicated ASIC/FPGA chip. In some embodiments, the localization image may contain a dedicated camera channel, by rendering 2D camera frames onto the 3D localization image using methods mentioned above, and optionally accelerated by a dedicated GPU. In some embodiments, the 2D camera frames may be captured from a camera sensor at the catheter's tip. In some embodiments, the raw camera images may be processed by an image signal processor (ISP) block 1112 in the ASIC/FPGA. In some embodiments, DeformNN module may be used for tracking the organ distortion in real-time using the rendered localization image 1116. In some embodiments, the system views are updated 1118 with the deformed localization images. In some embodiments, since the DeformNN module processes are computed on a dedicated ASIC/FPGA chip, the DeformNN data is delivered to the NavNN module 1016 for further use 1120. In some embodiments, following DeformNN actions, the NavNN can be executed to compute the best driving actions 1124 towards the target, or to stabilize the catheter on a moving target, similarly accelerated in hardware by the dedicated ASIC/FPGA. In some embodiments, the output from NavNN module is used to provide feedback 1122 to the operator, as described above. In some embodiments, once the target is reached (as realized by the high-level module 1022), optionally, a feedback can be given to the operator and biopsy and treatment tools can be inserted through a special working channel in the catheter. In some embodiments, additionally, before or after passing the localization image to the NavNN module, StressNN module may be used to estimate the force risk estimate of the catheter inside the lumen structure. In some embodiments, a force risk estimate close to 1 indicates that the catheter applies excessive force inside the lumen structure and the system may then stop, or the catheter may be automatically pulled back until relieved (as indicated by a force risk estimate close to 0 again). In some embodiments, the system flow is orchestrated by the microprocessor which can be a dedicated chip or can be incorporated as a block in the dedicated ASIC/FPGA chip.

In some embodiments, the wireless self-steering catheter can also be equipped with a WiFi instrument in order to transmit compressed (for example, using H.265) or uncompressed 2D/3D system views to an external monitor. In this case the views may be generated in real-time using the catheter's dedicated GPU and can be optionally encoded for example using a hardware accelerated H.265 encoder inside the ASIC/FPGA. In some embodiments, the system views can be displayed by any WiFi enabled device through a web-service, RTSP protocol a web browser or any other video streaming software. In some embodiments, the views are displayed on an external monitor, providing important 2D/3D navigational information for the operator physician or in a tablet or smartphone. In some embodiments, the endoscopic video can be displayed on a small portable display screen attached to the catheter's handle, similar to a periscopic or magnifying glass view. In some embodiments, the operator may then look “into the patient” through the small display, as if the catheter were a periscope. In some embodiments, the displayed endoscopic video may be augmented with additional 3D navigational data such as the pathway to target, the target or other navigational indications such as instructions to the physician, additional anatomical features from the CT (or MRI scan, or angiogram, etc.), etc. In some embodiments, additionally or alternatively, instead of displaying augmented endoscopic video views, pure virtual 3D views can be displayed, for example of the fully tracked catheter in its anatomical position inside the luminal structure, as displayed on an external monitor during common navigational procedures.

In some embodiments, the endoluminal device comprises one or more steering mechanisms configured to steer the endoluminal device toward one or more directions. In some embodiments, the one or more steering mechanisms comprise one or more of the following:

1. One or more pull wires: In some embodiments, the one or more wires are connected to one or more joints or points along the shaft.

2. One or more pre-curved shafts: In some embodiments, the one or more pre-curved are located one inside the other and where rotation of the pre-curved shafts one relative to the other causes deflection of the shaft, for example, when both curves of the shafts are aligned maximum deflection is achieved, while when the curves of the shafts are opposite to each other minimum deflection is achieved.

3. One or more shafts having different mechanical characteristics one within the other: In some embodiments, deflection of the shaft is achieved by using two shafts, where one is a pre-curved shaft and the other is not a pre-curved shaft and has variable stiffness. In some embodiments, deflection is performed by translating the shafts axially relative to each other —translation of the pre-curved section to a softer section of the variable stiffness shafts results in maximum deflection and translation of the pre-curved section to a stiffer section of the variable stiffness shafts results in minimum deflection.

4. A combination of either of the above to generate deflection, for example, where both shafts are pre-curved, have variable stiffness or both, with either rotation, axial translation or both.

5. Two or more coaxial tubes: In some embodiments, deflection of the shaft is performed by using two coaxial tubes where the stiffness of one tube is not uniform around the circumference of the cross section of the tube. In some embodiments, varying stiffness around the circumference of the cross section of the tube can be achieved by varying material composition and/or structure of the cross section, by selective removal of material around the circumference, or by combination thereof. In some embodiments, deflection is achieved by performing axial translation of the tubes one relative to the other, causing the shaft to deflect towards the softer side of the variable stiffness tube when it is in compression and towards the stiffer side of the tube when it is under tension.

In some embodiments, deflecting the shaft is performed by using one or more of the methods described above when both tubes have variable stiffness around the circumference and the tubes are assembled with the stiff sides in miss-alignment.

In some embodiments, deflecting the shaft is performed by using one or more of the methods described above (pre-curved shafts or variable stiffness around the circumference) in multiple sections by giving the shaft pre-curves or varying stiffness around the circumference in multiple sections. In some embodiments, pre-curve and varying stiffness or different sections can be aligned or in different orientations.

In some embodiments, steering actions are one or more of the following:
1. Rotation of shaft clockwise and counter clockwise.
2. Advancing forward or backward of shaft.
3. Deflecting the tip, for example by using one or more pull wires.
4. Uni-directional deflection: for example using a single pull wire.
5. Bi-directional deflection: for example by using two pull wires.
6. Multi-directional deflection: for example:
   i. by using more than 2 pull wires, for example 4 wires in two perpendicular plains, thereby allowing deflection and straightening in two plains in two directions in each plain, when pulling one wire per plain at a time while releasing the opposite wire;
   ii. by using more than 2 pull wires, for example 3 or 4 pull wires, distributed around the shaft axis, thereby allowing deflection in any direction by combination of pulling one or more wires.
7. Deflecting the tip using one or more pull wires to achieve out of plain 3 dimensional deflection:
   i. Dual pull wires in one plain where this plain is at an offset from the symmetry plain of the shaft allowing out of plain deflection in two directions where these directions are out of plain and not opposite to one another.
   ii. One or more pull wires connected to shaft with a non-uniform stiffness along the circumference of the cross section of the shaft. In some embodiments, varying stiffness around the circumference of the cross section of the tube can be achieved by varying material composition and/or structure of the cross section, by selective removal of material around the circumference, or by combination of these methods. In some embodiments, deflection direction is determined by the circumferential position of the pull wire compared to the stiffness distribution around the circumference.
   iii. Deflection in the method described above where the cross section of the shaft changes along its axis by either varying the directionality of the stiffness in the cross section and/or the overall stiffness of the cross section and/or the position of the pull wire in the cross section, creating different directions of deflection along the axis of the catheter and allowing 3 dimensional out of plain deflection.

### Exemplary Tap-to-Drive Interface

In some embodiments, the system comprises a user interface configured to allow a user to control electromechanically driven endoluminal devices by indicating a destination. In some embodiments, the endoluminal device is advanced using other driving methods, for example by applying magnetic fields to a magnet-fitted device, or for example by using pneumatic or hydraulic pressure to actuate a device. In some embodiments, an operator actuates the system to cause the tip of an instrument to be navigated to a position in the organ by indicating to the system the desired end-position and orientation of the instrument tip. In some embodiments, once the operator has indicated the desired destination to the system, the system is then triggered to maneuver and drive the instrument, using AI or other methods, such that the resulting position is in the requested location and orientation in the body. In some embodiments, safety mechanisms are installed to prevent unwanted movements.

In some embodiments, the operator marks the desired end location and orientation of the device, for example, by tapping on a point in a 3D map representing the endoluminal structure, for example displayed on a touchscreen. In some embodiments, this causes the system to maneuver the tip of a device to the appropriate destination location in the organ. In some embodiments, the same is achieved, for example, by clicking a mouse pointer on a location on a computer screen displaying a depiction of the anatomy, for example a CT slice (or MRI scan, or angiogram, etc.). In some embodiments, for example, the operator indicates the location to the system by choosing from a menu or other UI element a predetermined position, such as a lung bronchus bifurcation, or such as vascular bifurcation, such as an anatomical landmark, or such as a predetermined target or tagged location. In some embodiments, optionally, the destination location is automatically suggested by the system, such as a location which is automatically identified as a suspected lesion. In some embodiments, optionally, the operator indicates the destination by issuing a voice command. It is understood that these embodiments are provided as examples, and additional embodiments of the invention are possible within the scope of this invention.

In some embodiments, the system displays a curved planar reconstruction type view, which is generated by multiple segments of CT planes (or other imaging modalities) “stitched” together to form a continuous 2D view for example from trachea to target, in the case of the lungs; or for example from an entry port in the femoral artery to a target in the cerebral vascularity. In some embodiments, such a view, for example following a pre-planned pathway, allows the user to view the anatomical details as encoded in the imaging while concentrating on the path which leads to the target. In some embodiments, at each bifurcation, the view displays only the “correct” choice which will lead to the target. In some embodiments, taking the “wrong turn” is intuitively detectable as the tip of the navigating device leaves the displayed imaging plane. In some embodiments, optionally, a warning to the user may also be displayed in such a case. In some embodiments, this view may be used to indicate to the system the destination of the next segment of navigation. For example, directly to the target by pointing at it, or, for example, by having multiple waypoints at different points along the path, for example at each luminal bifurcation. In some embodiments, this potentially allows the operator to easily have a selection of “progress bar” style points to advance the device. In some embodiments, waypoints may be reached incrementally, where the user only instructs the system to proceed to the next waypoint, until reaching the target. In some embodiments, the view is compact and encodes all information relevant to the physician to supervise the semi-autonomous navigation process, including all surrounding anatomical features (as seen in the displayed CT strip or other imaging modality used) as well as the final target. In some embodiments, when a user indicates a destination, such indication may be to a position within the lumen, or to a position extra-luminally, or otherwise unsafe or precarious locations, the system warns, limits and/or prevents the navigation according to safety limits or other considerations. In some embodiments, such limitations may be fixed by the manufacturer and/or determined pre-operatively by the operator and/or may be set ad-hoc by the operator, for example by a confirmation message evoked as response to operator action. In some embodiments, such safety mechanisms are optionally configured or overridden given appropriate operator permissions. In some embodiments, for example, the system may interpret any point indicated on a graphical user interface to be endoluminal, thus matching a point indicated outside the lumen to the closest point inside the lumen, on the luminal tree. In this example, the system may then position the tip of the catheter such that it is oriented exactly towards the point indicated by the user outside the lumen. In this example the system may indicate the corrected position in comparison to the originally indicated position. In some embodiments, other indications may be made to notify the user that an alternative location has been chosen. In some embodiments, the system may display an enlargement of the targeted area, so that the user is able to point exactly where the tip destination and alignment. For example, this may be done using a “magnifying glass” style view, which is evoked once the user indicates a target destination. In some embodiments, this enlarged view then allows fine-tuning the requested position or this may be achieved by a “first person” style view to aid the operator in choosing the exact tip orientation, for example on a 3D render of a lesion.

In some embodiments, the system is triggered to stop the advancement according to predetermined maximum travelled distance. For example, the driven device is only allowed to travel a limited leg before waiting for additional operator command. In some embodiments, a final destination may be indicated but is carried out one leg at a time, so that greater control is exerted. In some embodiments, safety areas may be indicated on the 3D map, wherein automatic movement is allowed, but outside-of movement must be controlled manually.

In some embodiments, the interface is limited by a safety mechanism in the form of a dead-man-switch type control, in which motion of the device tip is only enabled as long as a trigger switch is engaged, with a spring-loaded action to disable it. Another embodiment of such switch may be a foot paddle, which allows movement only as long as it is depressed. In other embodiments, other methods of press-to-operate mechanisms are employed.

### Exemplary Use of System in Vascular Clinical Applications

In some embodiments, for example, the system is used in neurovascular cases of an acute ischemic stroke caused by large vessel occlusion (LVO), or in another case, for example, in a peripheral arterial occlusion case. In some embodiments, a revascularization device is introduced to perform thrombectomy, for example, a stent-assisted thrombectomy, or for example direct aspiration thrombectomy technique, using one or more devices, for example a guidewire, or a micro-catheter, or a reperfusion catheter, or a stent retriever, or other. In some embodiments, each is fitted with shape and location sensors in their respective distal sections, and each is connected back to the tracking device allowing simultaneous tracking of shape, location, force exerted on each other and the vessels, and allowing a display of real-time deformation of the anatomical structures such as artery, clot, surrounding tissue, etc. In another embodiment used for example in endovascular cases, the same is achieved by reconstructing the device's 3D shape from one or multiple fluoroscopic projections in near real-time, to track the device and its shape, location, force exerted on each other and the anatomical lumen, and allowing a display of real-time deformation of the anatomical structures such as artery, clot, surrounding tissue, etc. In some embodiments, reconstructing the device's 3D shape from fluoroscopic projections is performed by identifying the device's tip or full curve in multiple fluoroscopic 2D projections, identify the fluoroscope's location in some reference coordinate system, for example using optical fiducials, and finding the device's 3D location and/or shape by means of optimization, such that the back-projected 2D device's curves will fit the observed 2D curves from the fluoroscopic projections.

As used herein with reference to quantity or value, the term “about” means “within ±20% of”.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise. For example, the term “a compound” or “at least one compound” may include a plurality of compounds, including mixtures thereof.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Unless otherwise indicated, numbers used herein and any number ranges based thereon are approximations within the accuracy of reasonable measurement and rounding errors as understood by persons skilled in the art.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A method of generating a steering plan for a self-steering endoluminal system, comprising:
a. selecting a location accessible through one or more lumens in a digital endoluminal map to which a self-steering endoluminal device needs to reach; said digital endoluminal map based on a preoperative volumetric image;
b. generating navigational actions for said endoluminal device to reach said location;
c. assessing deformations to one or more lumens from said one or more lumens in said digital endoluminal map;
d. updating said digital endoluminal map according to said deformations;
e. updating said steering plan according to a result of said updating said digital endoluminal map while said self-steering endoluminal system is reaching said location.

2. The method according to claim 1, further comprising performing said navigational actions until reaching said location.

3. The method according to claim 1, wherein said updating said steering plan is performed in real-time.

4. The method according to claim 1, wherein said method further comprises assessing stress levels on said lumens caused by said navigational actions performed by said endoluminal device; and
wherein said method is performed until said stress levels are below a predetermined threshold.

5. (canceled)

6. The method according to claim 1, further comprising providing said plan to said self-steering endoluminal system.

7. The method according to claim 1, further comprising generating said digital endoluminal map comprising said one or more lumens based on an image; and
wherein said image is one or more of a CT scan, MRI scan and an angiogram.

8-9. (canceled)

10. The method according to claim 1, wherein said method further comprises one or more of the following:
wherein generating navigational actions comprises running a first simulation of said navigational actions;
wherein assessing deformations comprises running a second simulation of said deformations; and further comprising updating said digital endoluminal map according to said deformations simulated in said second simulation
wherein assessing stress levels comprises running a simulation of said stress levels; and further comprising updating said navigational actions to cause a reduction in said stress levels.

11-14. (canceled)

15. The method according to claim 1, wherein said assessing deformations further comprises assessing deformation caused by breathing, heartbeats and other causes external to the self-steering endoluminal system.

16. A self-steering endoluminal system, comprising:
a. an endoluminal device comprising a self-steerable elongated body;
b. a computer memory storage medium, comprising instructions for:
i. receiving a selection of a location accessible through one or more lumens in a digital endoluminal map to which a self-steering endoluminal device needs to reach; said digital endoluminal map based on a preoperative volumetric image:
ii. generating navigational actions for said endoluminal device to reach said location;
iii. assessing deformations to one or more lumens from said one or more lumens in said digital endoluminal map:
iv. updating said digital endoluminal map according to said deformations;
v. updating a steering plan according to a result of said updating said digital endoluminal map while said self-steering endoluminal system is reaching said location.

17. The system according to claim 16, wherein said computer memory storage medium comprises one or more of:
a. a Navigational module comprising instructions for generating navigational actions to be performed by said steerable elongated body of said endoluminal device to reach a desired location as selected in a digital endoluminal map;
b. a Deformation module comprising instructions for assessing deformations to one or more lumens;
c. a High-level module comprising instructions to receive information from one or more of said Navigational module and said Deformation module and actuate said steerable elongated body of said endoluminal device accordingly;
d. a Stress module comprising instructions for assessing stress levels on said lumens caused by said steerable elongated body of said endoluminal device.

18. The system according to claim 17, wherein said High-level module further comprises instructions to receive information from said Stress module and actuate said steerable elongated body of said endoluminal device accordingly.

19. The system according to claim 16, wherein said endoluminal device comprises one or more sensors and at least one external transmitter for monitoring a location of said endoluminal device during said navigational actions.

20. (canceled)

21. The system according to claim 17, wherein said Navigational module comprises instructions to perform one or more of:
a. generating navigational actions to be performed by said steerable elongated body of said endoluminal device to aid reaching a desired location as selected in a digital endoluminal map;
b. running a first simulation of said navigational actions.

22. The system according to claim 17, wherein said High-level module further comprises instructions to perform one or more of:
a. generating a steering plan based on said received information;
b. generating said digital endoluminal map comprising said one or more of lumens based on an image; wherein said image is one or more of a CT scan, MRI scan and an angiogram.

23-26. (canceled)

27. The system according to claim 16, wherein said Deformation module further comprises instructions to perform one or more of:
a. running a second simulation of said deformations;
b. updating said digital endoluminal map according to said deformations simulated in said second simulation.

28. (canceled)

29. The system according to claim 17, wherein said Stress module further comprises instructions to perform one or more of:
a. running a third simulation of said stress levels;
b. updating said navigational actions to cause a reduction in said stress levels.

30. (canceled)

31. The system according to claim 16, wherein said assessing deformations further comprises assessing deformation caused by breathing, heartbeats and other causes external to the self-steering endoluminal system.

32. The system according to claim 16, wherein said endoluminal device comprises one or more steering mechanisms comprising one or more pull wires, one or more pre-curved shafts, one or more shafts having variable stiffness along a body of said one or more shaft and one or more coaxial tubes;
wherein one or more of said one or more pre-curved shafts and one or more shafts having variable stiffness along a body of said one or more shaft are one within another; and
wherein said one or more steering mechanisms are configured to cause one or more steering actions comprising rotation of the shaft, advancing/retracting the shaft, deflection of the tip of the device and deflection of a part of the shaft of the device.

33-34. (canceled)

35. The system according to claim 16, wherein said computer memory storage medium further comprises instructions for tracking at least a partial curve of said endoluminal device.

36. The system according to claim 35, further comprising incorporating data from said tracking into said digital endoluminal map.

37. The system according to claim 35, further comprising assessing said deformations of said one or more lumens from said one or more lumens according to a result of said tracking of said at least a partial curve of said endoluminal device.

38. The system according to claim 35, further comprising updating said steering plan according to a result of said tracking of said at least a partial curve of said endoluminal device.

39. The system according to claim 35, further comprising updating said digital endoluminal map according to a result of said tracking of said at least a partial curve of said endoluminal device.

40. The system according to claim 35, further comprising assessing stress levels according to a result of said tracking of said at least a partial curve of said endoluminal device.

41. The system according to claim 18, wherein said actuate said steerable elongated body of said endoluminal device comprises actuate said steerable elongated body of said endoluminal device to cause a reduction in said stress levels.

42. The system according to claim 16, wherein said assessing deformations further comprises assessing deformation caused by said navigational actions performed by said endoluminal device.

43. The method according to claim 1, wherein said assessing deformations further comprises assessing deformation caused by said navigational actions performed by said endoluminal device.

44. The method according to claim 1, further comprising tracking at least a partial curve of said endoluminal device.

45. The method according to claim 44, further comprising incorporating data from said tracking into said digital endoluminal map.

46. The method according to claim 44, further comprising assessing said deformations of said to one or more lumens from said one or more lumens according to a result of said tracking of said at least a partial curve of said endoluminal device.

47. The method according to claim 44, further comprising updating said steering plan according to a result of said tracking of said at least a partial curve of said endoluminal device.

48. The method according to claim 44, further comprising updating said digital endoluminal map according to a result of said tracking of said at least a partial curve of said endoluminal device.

49. The method according to claim 4, further comprising assessing said stress levels by tracking at least a partial curve of said endoluminal device.

50. The method according to claim 4, further comprising actuating a steerable elongated body of said endoluminal device to cause a reduction in said stress levels.

51. A method of generating a steering action for a self-steering endoluminal system, comprising:
a. while said self-steering endoluminal system is reaching a selected location, assessing deformations to one or more lumens;
b. updating a digital endoluminal map according to said deformations; said digital endoluminal map based on a preoperative volumetric image;
c. generating a steering action for said endoluminal device according to said updated digital endoluminal map.
